SEQUENCE LISTING (140 sequences)

SEQ ID NO: 1 - protein, 119 aa, synthetic construct [sequence omitted]

SEQ ID NO: 2 - protein, 110 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVL

SEQ ID NO: 3 - protein, 5 aa, synthetic construct
DNWIG

SEQ ID NO: 4 - protein, 17 aa, synthetic construct
IIYPGDSDTRYSPSFQG

SEQ ID NO: 5 - protein, 10 aa, synthetic construct
LDWNYNPLRY

SEQ ID NO: 6 - protein, 13 aa, synthetic construct
SGSSSDIGSNYVS

SEQ ID NO: 7 - protein, 7 aa, synthetic construct
DNNKRPS

SEQ ID NO: 8 - protein, 11 aa, synthetic construct
GTWDSRLGIAV

SEQ ID NO: 9 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 10 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 11 - protein, 5 aa, synthetic construct
SYGMH

SEQ ID NO: 12 - protein, 17 aa, synthetic construct
IIWFDGSSTYYADSVRG

SEQ ID NO: 13 - protein, 9 aa, synthetic construct
ELGRRYFDL

SEQ ID NO: 14 - protein, 11 aa, synthetic construct
RASQSVSSYLA

SEQ ID NO: 15 - protein, 7 aa, synthetic construct
DASKRAT

SEQ ID NO: 16 - protein, 10 aa, synthetic construct
QQRSKWPPWT

SEQ ID NO: 17 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 18 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - protein, 107 aa, synthetic construct [sequence omitted]

SEQ ID NO: 21 - protein, 100 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 22 - protein, 105 aa, synthetic construct
SSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 108 aa, synthetic construct
ASGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 107 aa, synthetic construct
ASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 453 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSASGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 215 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 31 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 119 aa, synthetic construct
EVQLVQSGAEVKKPGASVKVSCKASGYSLTDNWIGWVRQAPGQGLEWMGIIYPGDSDTRYAQKFQGRVTMTADKSTSTVYMELSSLRSEDTAVYYCVGLDWNYNPLRYWGQGTLVTVSS

SEQ ID NO: 34 - protein, 110 aa, synthetic construct
QSVLTQPPSVSGAPGQRVTISCSGSSSDIGSNYVSWYQQLPGTAPKLLIYDNNKRPSGVPDRFSGSKSGTSASLAITGLQAEDEADYYCGTWDSRLGIAVFGGGTKLTVL

SEQ ID NO: 35 - protein, 119 aa, synthetic construct
EVQLVQSGAEVKKPGESLKISCKASGYELNDNWIGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 36 - protein, 119 aa, synthetic construct
EVQLVQSGAEVKKPGESLKISCKASGYQLNDNWIGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 37 - protein, 119 aa, synthetic construct
EVQLVQSGAEVKKPGESLKISCKASGYELKDNWIGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 38 - protein, 119 aa, synthetic construct
EVQLVQSGAEVKKPGESLKISCKASGYQLKDNWAGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 39 - protein, 119 aa, synthetic construct
EVQLVQSGAEVKKPGESLKISCKASGYQLKDNWIGWVRQKPGKGLEWMGIIYPGDSDTRYSPSFQGQVTISADKSISTAYLQWSSLKASDTAIYYCVGLEWNYNPLRYWGQGTLVTVSS

SEQ ID NO: 40 - protein, 119 aa, synthetic construct
EVQLVQSGAEVKKPGESLKISCKASGYQLKDNWIGWVRQKPGKGLEWMGIIYPGDSDTRYSPSFKGQVTISADKSISTAYLQWSSLKASDTAIYYCVGLEWNYNPGRYWGQGTLVTVSS

SEQ ID NO: 41 - protein, 119 aa, synthetic construct
EVQLVQSGAEVKKPGESLKISCKASGYQLKDNWAGWVRQKPGKGLEWMGIIYPGDSDTRYSPSFKGQVTISADKSISTAYLQWSSLKASDTAIYYCVGLEWNYNPGRYWGQGTLVTVSS

SEQ ID NO: 42 - protein, 119 aa, synthetic construct
EVQLVQSGAEVKKPGESLKISCKASGYQLKDNWIGWVRQKPGKGLEWMGIIYPGDSDTRYSPSFQGQVTISADKSISTAYLQWSSLKASDTAIYYCVGLEWNYNPVRYWGQGTLVTVSS

SEQ ID NO: 43 - protein, 119 aa, synthetic construct
EVQLVQSGAEVKKPGESLKISCKASGYQLKDNWIGWVRQKPGKGLEWMGIIYPGDSDTRYSPSFQGQVTISADKSISTAYLQWSSLKASDTAIYYCVGGEWNYNPLRYWGQGTLVTVSS

SEQ ID NO: 44 - protein, 119 aa, synthetic construct
EVQLVQSGAEVKKPGASVKVSCKASGYELNDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 45 - protein, 119 aa, synthetic construct
EVQLVQSGAEVKKPGASVKVSCKASGYQLNDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 46 - protein, 119 aa, synthetic construct
EVQLVQSGAEVKKPGASVKVSCKASGYELKDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 47 - protein, 119 aa, synthetic construct
EVQLVQSGAEVKKPGASVKVSCKASGYQLKDNWAGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 48 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 49 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGLEWNYNPGRYWGQGTLVTVSS

SEQ ID NO: 50 - protein, 119 aa, synthetic construct
EVQLVQSGAEVKKPGASVKVSCKASGYQLKDNWAGWVRQAPGQGLEWMGIIYPGDSDTRYAQKFQGRVTMTADKSTSTVYMELSSLRSEDTAVYYCVGLEWNYNPGRYWGQGTLVTVSS

SEQ ID NO: 51 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGLEWNYNPVRYWGQGTLVTVSS

SEQ ID NO: 52 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGGEWNYNPLRYWGQGTLVTVSS

SEQ ID NO: 53 - protein, 110 aa, synthetic construct
QSVLTQPPSVSAAPGQKVTISCSGSSSDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 54 - protein, 110 aa, synthetic construct
QSVLTQPPSVSAAPGQKVTISCSGSSTNIGALWVSWYQQLPGTAPKLLIYDNNKRPSGIPDRFSGSKSGTSATLGITGLQTGDEADYYCATWDSRFGIAVFGGGTQLTVL

SEQ ID NO: 55 - protein, 110 aa, synthetic construct
QSVLTQPPSVSAAPGQKVTISCSGSSTNIGALWVSWYQQLPGTAPKLLIYDNNKRPSGIPDRFSGSKSGTSATLGITGLQTGDEADYYCATWDSRWGIAVFGGGTQLTVL

SEQ ID NO: 56 - protein, 110 aa, synthetic construct
QSVLTQPPSVSAAPGQKVTISCSGSSTNIGANYVSWYQQLPGTAPKLLIYDNNKRPSGIPDRFSGSKSGTSATLGITGLQTGDEADYYCATWDSRFGIAVFGGGTQLTVL

SEQ ID NO: 57 - protein, 110 aa, synthetic construct
QSVLTQPPSVSAAPGQKVTISCSGSSTNIGANYVSWYQQLPGTAPKLLIYDNNKRPSGIPDRFSGSKSGTSATLGITGLQTGDEADYYCATWDSRWGIAVFGGGTQLTVL

SEQ ID NO: 58 - protein, 110 aa, synthetic construct
QSVLTQPPSVSAAPGQKVTISCSGSSTDIGANYVSWYQQLPGTAPKLLIYDNNKRPSGIPDRFSGSKSGTSATLGITGLQTGDEADYYCATWDSRWGIAVFGGGTQLTVL

SEQ ID NO: 59 - protein, 110 aa, synthetic construct
QSVLTQPPSVSAAPGQKVTISCSGSSTNIGANYVSWYQQLPGTAPKLLIYDNQKRPSGIPDRFSGSKSGTSATLGITGLQTGDEADYYCATWDSRWGIAVFGGGTQLTVL

SEQ ID NO: 60 - protein, 110 aa, synthetic construct
QSVLTQPPSVSGAPGQRVTISCSGSSTNIGALWVSWYQQLPGTAPKLLIYDNNKRPSGVPDRFSGSKSGTSASLAITGLQAEDEADYYCATWDSRFGIAVFGGGTKLTVL

SEQ ID NO: 61 - protein, 110 aa, synthetic construct
QSVLTQPPSVSGAPGQRVTISCSGSSTNIGALWVSWYQQLPGTAPKLLIYDNNKRPSGVPDRFSGSKSGTSASLAITGLQAEDEADYYCATWDSRWGIAVFGGGTKLTVL

SEQ ID NO: 62 - protein, 110 aa, synthetic construct
QSVLTQPPSVSGAPGQRVTISCSGSSTNIGANYVSWYQQLPGTAPKLLIYDNNKRPSGVPDRFSGSKSGTSASLAITGLQAEDEADYYCATWDSRFGIAVFGGGTKLTVL

SEQ ID NO: 63 - protein, 110 aa, synthetic construct
QSVLTQPPSVSGAPGQRVTISCSGSSTNIGANYVSWYQQLPGTAPKLLIYDNNKRPSGVPDRFSGSKSGTSASLAITGLQAEDEADYYCATWDSRWGIAVFGGGTKLTVL

SEQ ID NO: 64 - protein, 110 aa, synthetic construct
QSVLTQPPSVSGAPGQRVTISCSGSSTDIGANYVSWYQQLPGTAPKLLIYDNNKRPSGVPDRFSGSKSGTSASLAITGLQAEDEADYYCATWDSRWGIAVFGGGTKLTVL

SEQ ID NO: 65 - protein, 110 aa, synthetic construct
QSVLTQPPSVSGAPGQRVTISCSGSSTNIGANYVSWYQQLPGTAPKLLIYDNQKRPSGVPDRFSGSKSGTSASLAITGLQAEDEADYYCATWDSRWGIAVFGGGTKLTVL

SEQ ID NO: 66 - protein, 10 aa, synthetic construct
GYELNDNWIG

SEQ ID NO: 67 - protein, 10 aa, synthetic construct
GYQLNDNWIG

SEQ ID NO: 68 - protein, 10 aa, synthetic construct
GYELKDNWIG

SEQ ID NO: 69 - protein, 10 aa, synthetic construct
GYQLKDNWAG

SEQ ID NO: 70 - protein, 10 aa, synthetic construct
GYQLKDNWIG

SEQ ID NO: 71 - protein, 17 aa, synthetic construct
IIYPGDSDTRYSPSFKG

SEQ ID NO: 72 - protein, 12 aa, synthetic construct
VGLEWNYNPLRY

SEQ ID NO: 73 - protein, 12 aa, synthetic construct
VGLEWNYNPGRY

SEQ ID NO: 74 - protein, 12 aa, synthetic construct
VGLEWNYNPVRY

SEQ ID NO: 75 - protein, 12 aa, synthetic construct
VGGEWNYNPLRY

SEQ ID NO: 76 - protein, 13 aa, synthetic construct
SGSSTNIGALWVS

SEQ ID NO: 77 - protein, 13 aa, synthetic construct
SGSSTNIGANYVS

SEQ ID NO: 78 - protein, 13 aa, synthetic construct
SGSSTDIGANYVS

SEQ ID NO: 79 - protein, 7 aa, synthetic construct
DNQKRPS

SEQ ID NO: 80 - protein, 11 aa, synthetic construct
ATWDSRFGIAV

SEQ ID NO: 81 - protein, 11 aa, synthetic construct
ATWDSRWGIAV

SEQ ID NO: 82 - protein, 12 aa, synthetic construct
VGLDWNYDPLRY

SEQ ID NO: 83 - protein, 12 aa, synthetic construct
VGLEWNYNPLRY

SEQ ID NO: 84 - protein, 10 aa, synthetic construct
GYELTDNWIG

SEQ ID NO: 85 - protein, 10 aa, synthetic construct
GYQLTDNWIG

SEQ ID NO: 86 - protein, 10 aa, synthetic construct
GYSLNDNWIG

SEQ ID NO: 87 - protein, 10 aa, synthetic construct
GYSLKDNWIG

SEQ ID NO: 88 - protein, 10 aa, synthetic construct
GYSLTDNWTG

SEQ ID NO: 89 - protein, 18 aa, synthetic construct
IIYPGDSDTRYSPSFKGQ

SEQ ID NO: 90 - protein, 12 aa, synthetic construct
VGTDWNYNPLRY

SEQ ID NO: 91 - protein, 11 aa, synthetic construct
GTWDSRFGIAV

SEQ ID NO: 92 - protein, 13 aa, synthetic construct
SGSSSDIGANYVS

SEQ ID NO: 93 - protein, 11 aa, synthetic construct
GTWDSRLGIAV

SEQ ID NO: 94 - protein, 13 aa, synthetic construct
SGSSSDIGSLYVS

SEQ ID NO: 95 - protein, 11 aa, synthetic construct
GTWDSRLGIAV

SEQ ID NO: 96 - protein, 13 aa, synthetic construct
SGSSTDIGSNYVS

SEQ ID NO: 97 - protein, 13 aa, synthetic construct
SGSSSDIGSNWVS

SEQ ID NO: 98 - protein, 13 aa, synthetic construct
SGSSSNIGSNYVS

SEQ ID NO: 99 - protein, 11 aa, synthetic construct
ATWDSRLGIAV

SEQ ID NO: 100 - protein, 8 aa, synthetic construct
GYSLTDNW

SEQ ID NO: 101 - protein, 8 aa, synthetic construct
IYPGDSDT

SEQ ID NO: 102 - protein, 12 aa, synthetic construct
VGLDWNYNPLRY

SEQ ID NO: 103 - protein, 8 aa, synthetic construct
LSDIGSNY

SEQ ID NO: 104 - protein, 7 aa, synthetic construct
DNNKRPS

SEQ ID NO: 105 - protein, 11 aa, synthetic construct
GTWDSRLGIAV

SEQ ID NO: 106 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 107 - protein, 110 aa, synthetic construct
QSVLTQPPSVSAAPGQKVTISCSGSLSDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 108 - protein, 8 aa, synthetic construct
GYSLTDNW

SEQ ID NO: 109 - protein, 8 aa, synthetic construct
IYPGDSDT

SEQ ID NO: 110 - protein, 12 aa, synthetic construct
VGLDWNYNPLRY

SEQ ID NO: 111 - protein, 8 aa, synthetic construct
LRDIGSNY

SEQ ID NO: 112 - protein, 7 aa, synthetic construct
DNNKRPS

SEQ ID NO: 113 - protein, 11 aa, synthetic construct
GTWDSRLGIAV

SEQ ID NO: 114 - protein, 119 aa, synthetic construct; identical to SEQ ID NO: 106

SEQ ID NO: 115 - protein, 110 aa, synthetic construct
QSVLTQPPSVSAAPGQKVTISCSGSLRDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 116 - protein, 8 aa, synthetic construct
GYSLTDNW

SEQ ID NO: 117 - protein, 8 aa, synthetic construct
IYPGDSTT

SEQ ID NO: 118 - protein, 12 aa, synthetic construct
VGLDWNYNPLRY

SEQ ID NO: 119 - protein, 8 aa, synthetic construct
YSDIGSNY

SEQ ID NO: 120 - protein, 7 aa, synthetic construct
DNNKRPS

SEQ ID NO: 121 - protein, 11 aa, synthetic construct
GTWDSRLGIAV

SEQ ID NO: 122 - protein, 119 aa, synthetic construct
EVQLVQSGAEVKKPGASVKVSCKASGYSLTDNWIGWVRQAPGQGLEWMGIIYPGDSTTRYAQKFQGRVTMTADKSTSTVYMELSSLRSEDTAVYYCVGLDWNYNPLRYWGQGTLVTVSS

SEQ ID NO: 123 - protein, 110 aa, synthetic construct
QSVLTQPPSVSGAPGQRVTISCSGSYSDIGSNYVSWYQQLPGTAPKLLIYDNNKRPSGVPDRFSGSKSGTSASLAITGLQAEDEADYYCGTWDSRLGIAVFGGGTKLTVL

SEQ ID NO: 124 - protein, 8 aa, synthetic construct
GYSLTDNW

SEQ ID NO: 125 - protein, 8 aa, synthetic construct
IYPGDSDT

SEQ ID NO: 126 - protein, 12 aa, synthetic construct
VGLDWNYNPLRY

SEQ ID NO: 127 - protein, 8 aa, synthetic construct
LRDIGSNY

SEQ ID NO: 128 - protein, 7 aa, synthetic construct
DNNKRPS

SEQ ID NO: 129 - protein, 11 aa, synthetic construct
GTWDSRLGIAV

SEQ ID NO: 130 - protein, 119 aa, synthetic construct; identical to SEQ ID NO: 33

SEQ ID NO: 131 - protein, 110 aa, synthetic construct
QSVLTQPPSVSGAPGQRVTISCSGSLRDIGSNYVSWYQQLPGTAPKLLIYDNNKRPSGVPDRFSGSKSGTSASLAITGLQAEDEADYYCGTWDSRLGIAVFGGGTKLTVL

SEQ ID NO: 132 - protein, 8 aa, synthetic construct
GYSLTDNW

SEQ ID NO: 133 - protein, 8 aa, synthetic construct
IYPGDSDT

SEQ ID NO: 134 - protein, 12 aa, synthetic construct
VGLDWNYNPLRY

SEQ ID NO: 135 - protein, 8 aa, synthetic construct
SSNVGSNY

SEQ ID NO: 136 - protein, 7 aa, synthetic construct
DNNYVPS

SEQ ID NO: 137 - protein, 11 aa, synthetic construct
GTWDSRLDAAV

SEQ ID NO: 138 - protein, 119 aa, synthetic construct; identical to SEQ ID NO: 33

SEQ ID NO: 139 - protein, 110 aa, synthetic construct
QSVLTQPPSVSGAPGQRVTISCSGSSSNVGSNYVSWYQQLPGTAPKLLIYDNNYVPSGVPDRFSGSKSGTSASLAITGLQAEDEADYYCGTWDSRLDAAVFGGGTKLTVL

SEQ ID NO: 140 - protein, 6 aa, synthetic construct
KSCDKT

(19) United States
(12) Patent Application Publication
(10) Pub. No.: US 2026/0337284 A1
(43) Pub. Date: Oct. 29, 2026

(54) MULTISPECIFIC ANTIBODIES AND METHODS OF USE THEREOF

(71) Applicant: ETHYREAL BIO, INC., Cambridge, MA (US)

(72) Inventors: Charles Gordon Grummitt, Bagunte (PT); Simon Lempriere Westbrook, Dubai (AE); Lauren Pepper, Belmont, MA (US); Alexey Alexandrovich Lugovskoy, Belmont, MA (US); Melissa Geddie, Arlington, MA (US)

(21) Appl. No.: 19/305,435

(22) Filed: Aug. 20, 2025

Related U.S. Application Data
(60) Provisional application No. 63/685,037, filed on Aug. 20, 2024.

(51) Int. Cl. C07K 16/28

(52) U.S. Cl. CPC C07K 16/2869; C07K 16/2863; C07K 2317/31; C07K 2317/565; C07K 2317/92

(57) ABSTRACT

The present invention provides multispecific antibodies against insulin like growth factor I receptor (IGF-1R) and thyroid stimulating hormone (TSH) receptor (TSHR), methods for their production, pharmaceutical compositions containing the multispecific antibodies, and uses thereof. More particularly, the presently disclosed invention provides bispecific IGF-IR and TSHR antibodies, and antigen binding fragments thereof.

### RELATED APPLICATIONS

This application claims the benefit of U.S. Provisional Application Ser. No. 63/685,037, filed Aug. 20, 2024, the entire disclosure of which is incorporated herein by reference.

### SEQUENCE LISTING

The instant application contains a Sequence listing which has been submitted electronically in XML format and is herein incorporated by reference in its entirety. Said XML copy, created on Oct. 8, 2025, is named 73083-704_201_SL.xml and is 130,377 bytes in size.

### BACKGROUND

Current therapies for hyperthyroidism due to Graves' disease are imperfect because therapies targeting the specific underlying pathogenic autoimmune mechanisms of the disease are lacking. Even more complex is the treatment of moderate-to-severe active TAO. Although recent years have witnessed a better understanding of its pathogenesis, TAO remains a therapeutic challenge and dilemma. Intravenous glucocorticoids (ivGCs) and oral glucocorticoids are used to treat patients with moderate-to-severe active TAO, but results are seldom satisfactory. Partial responses are frequent and relapses (rebound) after drug withdrawal are not uncommon. The only approved drug to treat active TAO is IGF-1R monoclonal antibody teprotumumab-trbw (TEPEZZA®) (International patent application published as WO2005005635, Smith T J, et al., Teprotumumab for Thyroid-Associated Ophthalmopathy. N Engl J Med. 2017 May 4; 376 (18): 1748-1761. doi: 10.1056/NEJMoa1614949, and | International patent application published as WO2021041733. Adverse events do occur and many patients eventually require rehabilitative surgery conducted when their condition has transitioned to inactive TAO. Accordingly, there is still a need to provide alternative therapies for TAO and its related symptoms.

### SUMMARY OF THE INVENTION

The present invention provides multispecific antibodies against insulin like growth factor I receptor (IGF-1R) and thyroid stimulating hormone (TSH) receptor (TSHR), methods for their production, pharmaceutical compositions containing the multispecific antibodies, and uses thereof. More particularly, the presently disclosed invention provides bispecific IGF-IR and TSHR antibodies, and antigen binding fragments thereof. Certain multispecific antibodies and antigen-binding fragments inhibit IGF-1R function or block the biological functions of IGF-1 mediated IGF-1R signalling and inhibit TSHR function or block the biological functions of TSH mediated TSHR signalling.

The present invention provides, inter alia, multispecific antibody molecules that are capable of binding to IGF-1R and TSHR.

In one aspect, the present invention provides a multispecific antibody comprising at least one anti-TSHR antibody or antigen-binding fragment thereof and at least one anti-TSHR antibody or antigen-binding fragment thereof. In one embodiment, the multispecific antibody is a bispecific antibody.

In one embodiment, the at least one anti-TSHR antibody or antigen-binding fragment thereof of the multispecific antibody has a higher affinity to TSHR than the at least one anti-IGF-1R antibody or antigen-binding fragment thereof of the multispecific antibody has affinity to IGF-1R. In a further embodiment the at least one anti-TSHR antibody or antigen-binding fragment thereof has an about a 5 fold to about a 1,000 fold higher affinity to TSHR than the at least one anti-IGF-1R antibody or antigen-binding fragment thereof has affinity to IGF-1R as determined by the dissociation equilibrium constant K (Kd). In a further embodiment the at least one anti-TSHR antibody or antigen-binding fragment thereof has an about a 5 fold to about a 100 fold higher affinity to TSHR than the at least one anti-IGF-1R antibody or antigen-binding fragment thereof has affinity to IGF-1R as determined by the dissociation equilibrium constant K (Kd). In a further embodiment the at least one anti-TSHR antibody or antigen-binding fragment thereof has an about a 5 fold to about a 50 fold higher affinity to TSHR than the at least one anti-IGF-1R antibody or antigen-binding fragment thereof has affinity to IGF-1R as determined by the dissociation equilibrium constant K (Kd). In a further embodiment the at least one anti-TSHR antibody or antigen-binding fragment thereof has an about a 5 fold to about a 10 fold higher affinity to TSHR than the at least one anti-IGF-1R antibody or antigen-binding fragment thereof has affinity to IGF-1R as determined by the dissociation equilibrium constant K (Kd). In a further embodiment the at least one anti-TSHR antibody or antigen-binding fragment thereof has an at least 5 fold, at least 10 fold, or at least 100 fold, higher affinity to TSHR than the at least one anti-IGF-1R antibody or antigen-binding fragment thereof has affinity to IGF-1R as determined by the dissociation equilibrium constant K (Kd). In particular the multispecific antibody has TSHR affinity with dissociation equilibrium constant K (Kd) in the pM range and IGF-1R affinity in the nM range.

Whilst not bound by theory, IGF-1R is widely expressed and as a result the currently available TED drug which targets solely IGF-1R, Tepezza®, has a number of adverse events including hyperglycemia, muscle cramps and potentially hearing loss (Douglas R S, et al., Teprotumumab Efficacy, Safety, and Durability in Longer-Duration Thyroid Eye Disease and Re-treatment: OPTIC-X Study. Ophthalmology. 2022 April; 129 (4): 438-449; Douglas R S, et al., Teprotumumab for the Treatment of Active Thyroid Eye Disease. N Engl J Med. 2020 Jan. 23; 382 (4): 341-352). Conversely TSHR is not widely expressed and is only found on a relatively small number of cell types. It is therefore an advantage of the present invention that the multispecific antibody with higher affinity to TSHR than IGF-1R may reduce the widespread systemic binding and resulting adverse events observed with Tepezza and increase specificity to the cells of interest in relation to TED, particularly the Graves Orbital Fibroblasts which co-express both receptors.

The inventors have identified the sequences of multispecific antibodies to IGF-1R and TSHR. Those sequences and uses for such antibodies are disclosed herein.

In particular, the present invention provides a multispecific antibody comprising at least one anti-TSHR antibody or antigen-binding fragment thereof, which binds specifically to TSHR, and at least one anti-IGF-1R antibody or antigen-binding fragment thereof, which binds specifically to IGF-1R; wherein the at least one anti-TSHR antibody or antigen-binding fragment thereof is as disclosed in PCT patent application WO2010/073012, in particular K1-70 or antigen-binding fragment thereof and wherein the at least one anti-IGF-1R antibody or antigen-binding fragment thereof is as disclosed in PCT patent application WO2005/005635 or U.S. Pat. No. 11,208,489, in particular teprotumumab or antigen-binding fragment thereof. In one embodiment, the at least one anti-TSHR antibody or antigen-binding fragment thereof comprises a heavy chain variable domain with a CDR1 of SEQ ID NO: 3, a CDR2 of SEQ ID NO: 4, and a CDR3 selected from the group consisting of SEQ ID NO: 5, and a light chain variable domain with a CDR1 of SEQ ID NO: 6, a CDR2 of SEQ ID NO: 7, and a CDR3 of SEQ ID NO: 8; and wherein the at least one anti-IGF-1R antibody or antigen-binding fragment thereof comprises a heavy chain variable domain with a CDR1 of SEQ ID NO: 11, a CDR2 of SEQ ID NO: 12, and a CDR3 of SEQ ID NO: 13, and a light chain variable domain with a CDR1 of SEQ ID NO: 14, a CDR2 of SEQ ID NO: 15, and a CDR3 of SEQ ID NO: 16.

In some embodiments, the anti-TSHR antibody, or antigen binding fragment thereof, comprising a sequence as provided for herein is provided. In some embodiments, the anti-TSHR antibody, or antigen binding fragment thereof, comprises a VH sequence as set forth in SEQ ID NO: 1; and a VL sequence as set forth in SEQ ID NO: 2.

In some embodiments, the anti-TSHR antibody, or antigen binding fragment thereof, comprises a VH peptide as set forth in SEQ ID NO: 1. In some embodiments, the anti-TSHR antibody, or antigen binding fragment thereof, comprises a VL peptide as set forth in SEQ ID NO: 2,

In some embodiments, the anti-TSHR antibody or antibody fragment comprises: (i) a heavy chain variable region comprising heavy chain CDR1, CDR2, and CDR3 sequences, wherein the heavy chain CDR1 sequence has the amino acid sequence of SEQ ID NO: 3; the heavy chain CDR2 has the amino acid sequence of SEQ ID NO: 4; and the heavy chain CDR3 sequence has the amino acid sequence of SEQ ID NO: 5; and (ii) a light chain variable region comprising light chain CDR1 CDR2, and CDR3 sequences, wherein the light chain CDR1 sequence has the amino acid sequence SEQ ID NO: 6; the light chain CDR2 sequence has the amino acid sequence of SEQ ID NO: 7; and the light chain CDR3 sequence has the amino acid sequence of SEQ ID NO: 8.

In some embodiments, the anti-IGF-1R antibody, or antigen binding fragment thereof, comprising a sequence as provided for herein is provided. In some embodiments, the anti-IGF-1R antibody, or antigen binding fragment thereof, comprises a VH sequence as set forth in SEQ ID NO: 9; and a VL sequence as set forth in SEQ ID NO: 10. In some embodiments, the anti-IGF-1R antibody, or antigen binding fragment thereof, comprises a VH peptide as set forth in SEQ ID NO: 9. In some embodiments, the anti-IGF-1R antibody, or antigen binding fragment thereof, comprises a VL peptide as set forth in SEQ ID NO: 10.

In some embodiments, the anti-IGF-1R antibody or antibody fragment comprises: (i) a heavy chain variable region comprising heavy chain CDR1, CDR2, and CDR3 sequences, wherein the heavy chain CDR1 sequence has the amino acid sequence of SEQ ID NO: 11; the heavy chain CDR2 has the amino acid sequence of SEQ ID NO: 128; and the heavy chain CDR3 sequence has the amino acid sequence of SEQ ID NO: 13; and (ii) a light chain variable region comprising light chain CDR1 CDR2, and CDR3 sequences, wherein the light chain CDR1 sequence has the amino acid sequence SEQ ID NO: 13; the light chain CDR2 sequence has the amino acid sequence of SEQ ID NO: 15; and the light chain CDR3 sequence has the amino acid sequence of SEQ ID NO: 16.

In some embodiments, the variant of any antibodies provided herein are provided so long as the CDRs remain constant as compared to the parental (non-variant) sequence provided for herein.

In some embodiments, the anti-TSHR antibody or antibody fragment comprises a light chain having a constant lambda region CA comprising an amino acid sequence of SEQ ID NO: 19 and a heavy chain having a constant region CH1 comprising an amino acid sequence of SEQ ID NO: 17.

In some embodiments, the anti-TSHR antibody or antibody fragment comprises a light chain having a constant kappa region CK having an amino acid sequence of SEQ ID NO: 20 and a heavy chain having a constant region CH1 comprising an amino acid sequence of SEQ ID NO: 17.

In some embodiments, the anti-TSHR antibody or antibody fragment comprises a light chain having a constant lambda region CA comprising an amino acid sequence of SEQ ID NO: 19 and a heavy chain having a constant region CH1 comprising an amino acid sequence of SEQ ID NO: 18.

In some embodiments, the anti-TSHR antibody or antibody fragment comprises a light chain having a constant kappa region CK having an amino acid sequence of SEQ ID NO: 20 and a heavy chain having a constant region CH1 comprising an amino acid sequence of SEQ ID NO: 18.

In some embodiments, the anti-IGF-1R antibody or antibody fragment comprises a light chain having a constant lambda region CA comprising an amino acid sequence of SEQ ID NO: 19 and a heavy chain having a constant region CH1 comprising an amino acid sequence of SEQ ID NO: 17.

In some embodiments, the anti-IGF-1R antibody or antibody fragment comprises a light chain having a constant kappa region CK having an amino acid sequence of SEQ ID NO: 20 and a heavy chain having a constant region CH1 comprising an amino acid sequence of SEQ ID NO: 17.

In some embodiments, the anti-IGF-1R antibody or antibody fragment comprises a light chain having a constant lambda region CA comprising an amino acid sequence of SEQ ID NO: 19 and a heavy chain having a constant region CH1 comprising an amino acid sequence of SEQ ID NO: 18.

In some embodiments, the anti-IGF-1R antibody or antibody fragment comprises a light chain having a constant kappa region CK having an amino acid sequence of SEQ ID NO: 20 and a heavy chain having a constant region CH1 comprising an amino acid sequence of SEQ ID NO: 18.

In some embodiments, the CH and CL (CA or CK) constant regions are exchanged.

In some embodiments, the anti-TSHR antibody or antibody fragment comprises a heavy chain having a constant lambda region CA comprising an amino acid sequence of SEQ ID NO: 23 and a light chain having a constant region CH1 comprising an amino acid sequence of SEQ ID NO: 21.

In some embodiments, the anti-TSHR antibody or antibody fragment comprises a heavy chain having a constant kappa region CK having an amino acid sequence of SEQ ID NO: 24 and a light chain having a constant region CH1 comprising an amino acid sequence of SEQ ID NO: 21.

In some embodiments, the anti-TSHR antibody or antibody fragment comprises a heavy chain having a constant lambda region CA comprising an amino acid sequence of SEQ ID NO: 23 and a light chain having a constant region CH1 comprising an amino acid sequence of SEQ ID NO: 22.

In some embodiments, the anti-TSHR antibody or antibody fragment comprises a heavy chain having a constant kappa region CK having an amino acid sequence of SEQ ID NO: 24 and a light chain having a constant region CH1 comprising an amino acid sequence of SEQ ID NO: 22.

In some embodiments, the anti-IGF-1R antibody or antibody fragment comprises a heavy chain having a constant lambda region CA comprising an amino acid sequence of SEQ ID NO: 23 and a light chain having a constant region CH1 comprising an amino acid sequence of SEQ ID NO: 21.

In some embodiments, the anti-IGF-1R antibody or antibody fragment comprises a heavy chain having a constant kappa region CK having an amino acid sequence of SEQ ID NO: 24 and a light chain having a constant region CH1 comprising an amino acid sequence of SEQ ID NO: 21.

In some embodiments, the anti-IGF-1R antibody or antibody fragment comprises a heavy chain having a constant lambda region CA comprising an amino acid sequence of SEQ ID NO: 23 and a light chain having a constant region CH1 comprising an amino acid sequence of SEQ ID NO: 22.

In some embodiments, the anti-IGF-1R antibody or antibody fragment comprises a heavy chain having a constant kappa region CK having an amino acid sequence of SEQ ID NO: 24 and a light chain having a constant region CH1 comprising an amino acid sequence of SEQ ID NO: 22.

In some embodiments, the CH and CL (CA or CK) constant regions are replaced by T cell receptor (TCR) constant regions.

In some embodiments, the multispecific antibody comprises a Fc region. In some embodiments, the Fc region is as set forth in SEQ ID NO: 25 or 26.

In some embodiments the Fc region comprises ‘knobs in holes’ (KiH) mutations in the CH3 domain, in particular a T366W mutation creates a knob whilst T366'S/L368′A/Y407′V mutations create complementary knob/hole pairings. In a yet further embodiment, further CH3 stabilising cysteines S354C (knob) and Y349C (hole) mutations are present.

In some embodiments, the Fc region is as set forth in paired sequences SEQ ID NO: 27, and 28.

In one embodiment, the present invention provides a multispecific antibody molecule, wherein the anti-TSHR antibody or antibody fragment is characterized by clone-paired heavy and light chain CDR sequences from Tables 2 and 3, respectively. The anti-TSHR antibody or antibody fragment may comprise light and heavy chain variable sequences according to clone-paired sequences from Table 1, or light and heavy chain variable sequences having at least 70%, 80%, 90% or 95% identity to clone-paired sequences from Table 1.

In one embodiment, the present invention provides a multispecific antibody molecule, wherein the anti-IGF-1R antibody or antibody fragment is characterized by clone-paired heavy and light chain CDR sequences from Tables 5 and 6, respectively. The anti-IGF-1R antibody or antibody fragment may comprise light and heavy chain variable sequences according to clone-paired sequences from Table 4, or light and heavy chain variable sequences having at least 70%, 80%, 90% or 95% identity to clone-paired sequences from Table 4.

In one embodiment, the at least one anti-TSHR antibody or antibody fragment comprises a variable heavy chain (VH) comprising an HCDR1 amino acid sequence of SEQ ID NO: 3, an HCDR2 amino acid sequence of SEQ ID NO: 4, and an HCDR3 amino acid sequence of SEQ ID NO: 5, and a variable light chain (VL) comprising an LCDR1 amino acid sequence of SEQ ID NO: 6, an LCDR2 amino acid sequence of SEQ ID NO: 7, and an LCDR3 amino acid sequence of SEQ ID NO: 8.

In one embodiment, the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 1, and a VL amino acid sequence comprising at least 90% identity (i.e., 90%, 91%, 92%, 93%, 94%, 95%, 96%, 97%, 98%, 99%, or 100% identity) to SEQ ID NO: 2.

In one embodiment, the at least one anti-TSHR antibody or antibody fragment comprises a variable heavy chain (VH) comprising an HCDR1 amino acid sequence of SEQ ID NO: 100, an HCDR2 amino acid sequence of SEQ ID NO: 101, and an HCDR3 amino acid sequence of SEQ ID NO: 102, and a variable light chain (VL) comprising an LCDR1 amino acid sequence of SEQ ID NO: 103, an LCDR2 amino acid sequence of SEQ ID NO: 104, and an LCDR3 amino acid sequence of SEQ ID NO: 105.

In one embodiment, the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 106, and a VL amino acid sequence comprising at least 90% identity (i.e., 90%, 91%, 92%, 93%, 94%, 95%, 96%, 97%, 98%, 99%, or 100% identity) to SEQ ID NO: 107.

In one embodiment, the at least one anti-TSHR antibody or antibody fragment comprises a variable heavy chain (VH) comprising an HCDR1 amino acid sequence of SEQ ID NO: 108, an HCDR2 amino acid sequence of SEQ ID NO: 109, and an HCDR3 amino acid sequence of SEQ ID NO: 110, and a variable light chain (VL) comprising an LCDR1 amino acid sequence of SEQ ID NO: 111, an LCDR2 amino acid sequence of SEQ ID NO: 112, and an LCDR3 amino acid sequence of SEQ ID NO: 113.

In one embodiment, the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 114, and a VL amino acid sequence comprising at least 90% identity (i.e., 90%, 91%, 92%, 93%, 94%, 95%, 96%, 97%, 98%, 99%, or 100% identity) to SEQ ID NO: 115.

In one embodiment, the at least one anti-TSHR antibody or antibody fragment comprises a variable heavy chain (VH) comprising an HCDR1 amino acid sequence of SEQ ID NO: 116, an HCDR2 amino acid sequence of SEQ ID NO: 117, and an HCDR3 amino acid sequence of SEQ ID NO: 118, and a variable light chain (VL) comprising an LCDR1 amino acid sequence of SEQ ID NO: 119, an LCDR2 amino acid sequence of SEQ ID NO: 120, and an LCDR3 amino acid sequence of SEQ ID NO: 121.

In one embodiment, the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 122, and a VL amino acid sequence comprising at least 90% identity (i.e., 90%, 91%, 92%, 93%, 94%, 95%, 96%, 97%, 98%, 99%, or 100% identity) to SEQ ID NO: 123.

In one embodiment, the at least one anti-TSHR antibody or antibody fragment comprises a variable heavy chain (VH) comprising an HCDR1 amino acid sequence of SEQ ID NO: 124, an HCDR2 amino acid sequence of SEQ ID NO: 125, and an HCDR3 amino acid sequence of SEQ ID NO: 126, and a variable light chain (VL) comprising an LCDR1 amino acid sequence of SEQ ID NO: 127, an LCDR2 amino acid sequence of SEQ ID NO: 128, and an LCDR3 amino acid sequence of SEQ ID NO: 129.

In one embodiment, the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 130, and a VL amino acid sequence comprising at least 90% identity (i.e., 90%, 91%, 92%, 93%, 94%, 95%, 96%, 97%, 98%, 99%, or 100% identity) to SEQ ID NO: 131.

In one embodiment, the at least one anti-TSHR antibody or antibody fragment comprises a variable heavy chain (VH) comprising an HCDR1 amino acid sequence of SEQ ID NO: 132, an HCDR2 amino acid sequence of SEQ ID NO: 133, and an HCDR3 amino acid sequence of SEQ ID NO: 134, and a variable light chain (VL) comprising an LCDR1 amino acid sequence of SEQ ID NO: 135, an LCDR2 amino acid sequence of SEQ ID NO: 136, and an LCDR3 amino acid sequence of SEQ ID NO: 137.

In one embodiment, the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 138, and a VL amino acid sequence comprising at least 90% identity (i.e., 90%, 91%, 92%, 93%, 94%, 95%, 96%, 97%, 98%, 99%, or 100% identity) to SEQ ID NO: 139.

In one embodiment, the at least one anti-TSHR antibody or antibody fragment is characterized by clone-paired heavy and light chain CDR sequences from Table 1C.

In one embodiment, the at least one anti-TSHR antibody or antibody fragment comprises light and heavy chain variable sequences according to clone-paired sequences from Table 1B.

In one embodiment, the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, a) the at least one anti-TSHR antibody or antibody fragment comprises a variable heavy chain (VH) comprising an HCDR1 amino acid sequence of SEQ ID NO: 3, an HCDR2 amino acid sequence of SEQ ID NO: 4, and an HCDR3 amino acid sequence of SEQ ID NO: 5, and a variable light chain (VL) comprising an LCDR1 amino acid sequence of SEQ ID NO: 6, an LCDR2 amino acid sequence of SEQ ID NO: 7, and an LCDR3 amino acid sequence of SEQ ID NO: 8; and b) the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, a) the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 1, and a VL amino acid sequence comprising at least 90% identity (i.e., 90%, 91%, 92%, 93%, 94%, 95%, 96%, 97%, 98%, 99%, or 100% identity) to SEQ ID NO: 2; and b) the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, a) the at least one anti-TSHR antibody or antibody fragment comprises a variable heavy chain (VH) comprising an HCDR1 amino acid sequence of SEQ ID NO: 100, an HCDR2 amino acid sequence of SEQ ID NO: 101, and an HCDR3 amino acid sequence of SEQ ID NO: 102, and a variable light chain (VL) comprising an LCDR1 amino acid sequence of SEQ ID NO: 103, an LCDR2 amino acid sequence of SEQ ID NO: 104, and an LCDR3 amino acid sequence of SEQ ID NO: 105; and b) the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, a) the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 106, and a VL amino acid sequence comprising at least 90% identity (i.e., 90%, 91%, 92%, 93%, 94%, 95%, 96%, 97%, 98%, 99%, or 100% identity) to SEQ ID NO: 107; and b) the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, a) the at least one anti-TSHR antibody or antibody fragment comprises a variable heavy chain (VH) comprising an HCDR1 amino acid sequence of SEQ ID NO: 108, an HCDR2 amino acid sequence of SEQ ID NO: 109, and an HCDR3 amino acid sequence of SEQ ID NO: 110, and a variable light chain (VL) comprising an LCDR1 amino acid sequence of SEQ ID NO: 111, an LCDR2 amino acid sequence of SEQ ID NO: 112, and an LCDR3 amino acid sequence of SEQ ID NO: 113; and b) the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, a) the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 114, and a VL amino acid sequence comprising at least 90% identity (i.e., 90%, 91%, 92%, 93%, 94%, 95%, 96%, 97%, 98%, 99%, or 100% identity) to SEQ ID NO: 115; and b) the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, a) the at least one anti-TSHR antibody or antibody fragment comprises a variable heavy chain (VH) comprising an HCDR1 amino acid sequence of SEQ ID NO: 116, an HCDR2 amino acid sequence of SEQ ID NO: 117, and an HCDR3 amino acid sequence of SEQ ID NO: 118, and a variable light chain (VL) comprising an LCDR1 amino acid sequence of SEQ ID NO: 119, an LCDR2 amino acid sequence of SEQ ID NO: 120, and an LCDR3 amino acid sequence of SEQ ID NO: 121; and b) the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, a) the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 122, and a VL amino acid sequence comprising at least 90% identity (i.e., 90%, 91%, 92%, 93%, 94%, 95%, 96%, 97%, 98%, 99%, or 100% identity) to SEQ ID NO: 123; and b) the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, a) the at least one anti-TSHR antibody or antibody fragment comprises a variable heavy chain (VH) comprising an HCDR1 amino acid sequence of SEQ ID NO: 124, an HCDR2 amino acid sequence of SEQ ID NO: 125, and an HCDR3 amino acid sequence of SEQ ID NO: 126, and a variable light chain (VL) comprising an LCDR1 amino acid sequence of SEQ ID NO: 127, an LCDR2 amino acid sequence of SEQ ID NO: 128, and an LCDR3 amino acid sequence of SEQ ID NO: 129; and b) the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, a) the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 130, and a VL amino acid sequence comprising at least 90% identity (i.e., 90%, 91%, 92%, 93%, 94%, 95%, 96%, 97%, 98%, 99%, or 100% identity) to SEQ ID NO: 131; and b) the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, a) the at least one anti-TSHR antibody or antibody fragment comprises a variable heavy chain (VH) comprising an HCDR1 amino acid sequence of SEQ ID NO: 132, an HCDR2 amino acid sequence of SEQ ID NO: 133, and an HCDR3 amino acid sequence of SEQ ID NO: 134, and a variable light chain (VL) comprising an LCDR1 amino acid sequence of SEQ ID NO: 135, an LCDR2 amino acid sequence of SEQ ID NO: 136, and an LCDR3 amino acid sequence of SEQ ID NO: 137; and b) the at least one anti-IGF-1R antibody or antigen-binding fragment thereof comprises a variable heavy chain (VH) comprising an HCDR1 amino acid sequence of SEQ ID NO: 11, an HCDR2 amino acid sequence of SEQ ID NO: 12, and an HCDR3 amino acid sequence of SEQ ID NO: 13, and a variable light chain (VL) comprising an LCDR1 amino acid sequence of SEQ ID NO: 14, an LCDR2 amino acid sequence of SEQ ID NO: 15, and an LCDR3 amino acid sequence of SEQ ID NO: 16.

In one embodiment, a) the at least one anti-TSHR antibody or antibody fragment comprises a VH amino acid sequence comprising at least 90% identity (i.e., 90%, 91%, 92%, 93%, 94%, 95%, 96%, 97%, 98%, 99%, or 100% identity) to SEQ ID NO: 138, and a VL amino acid sequence comprising at least 90% identity (i.e., 90%, 91%, 92%, 93%, 94%, 95%, 96%, 97%, 98%, 99%, or 100% identity) to SEQ ID NO: 139; and b) the at least one anti-IGF-1R antibody or antigen-binding fragment thereof comprises a VH amino acid sequence comprising at least 90% identity (i.e., 90%, 91%, 92%, 93%, 94%, 95%, 96%, 97%, 98%, 99%, or 100% identity) to SEQ ID NO: 9, and a VL amino acid sequence comprising at least 90% identity (i.e., 90%, 91%, 92%, 93%, 94%, 95%, 96%, 97%, 98%, 99%, or 100% identity) to SEQ ID NO: 10.

In one embodiment, the multispecific antibody fragment is a recombinant scFv (single chain fragment variable) antibody, Fab fragment, F(ab′)2 fragment, or Fv fragment.

In one embodiment, the multispecific antibody is an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

In one aspect, the disclosure provides a pharmaceutical composition comprising a multispecific antibody molecule described herein and one or more pharmaceutically acceptable excipients, diluents and/or carriers.

In one aspect, the disclosure provides a multispecific antibody molecule described herein for use as a medicament.

In one aspect, the disclosure provides a multispecific antibody molecule described herein for use in treating a condition or disorder in which aberrant stimulation of TSHR and/or aberrant signalling of IGF-1R occurs.

In one embodiment, the condition or disorder is thyroid-associated ophthalmopathy (TAO).

In one aspect, the disclosure provides a method of treating a condition or disorder in which aberrant stimulation of TSHR and/or aberrant signalling of IGF-1R occurs, comprising delivering to the subject a multispecific antibody molecule described herein.

In one embodiment, the condition or disorder is thyroid-associated ophthalmopathy (TAO).

To be used in therapy, the antibody molecule is included into pharmaceutical compositions appropriate to facilitate administration to animals or humans.

In some embodiments, pharmaceutical compositions comprising a multispecific antibody molecule as provided for herein is provided.

In a further aspect, the present invention provides a multispecific antibody molecule of the present invention for use as a medicament.

In one aspect, the present invention provides a method of treating a condition or disorder in which aberrant stimulation of TSHR and/or aberrant signalling of IGF-IR occurs, comprising delivering to the subject a multispecific antibody of the present invention. In particular, the multispecific antibody comprises an anti-TSHR antibody or antibody fragment having clone-paired heavy and light chain CDR sequences from Tables 2 and 3, respectively. The anti-TSHR antibody or antibody fragment may comprise light and heavy chain variable sequences according to clone-paired sequences from Table 1, or light and heavy chain variable sequences having at least 70%, 80%, 90% or 95% identity to clone-paired sequences from Table 1. In particular, the multispecific antibody comprises an anti-IGF-1R antibody or antibody fragment having clone-paired heavy and light chain CDR sequences from Tables 5 and 6, respectively. The anti-IGF-1R antibody or antibody fragment may comprise light and heavy chain variable sequences according to clone-paired sequences from Table 1, or light and heavy chain variable sequences having at least 70%, 80%, 90% or 95% identity to clone-paired sequences from Table 1.

The multispecific antibody fragment may be a recombinant scFv (single chain fragment variable) antibody, Fab fragment, F(ab′h fragment, or Fv fragment. The antibody may be a bispecific antibody. The multispecific antibody may be an IgG, or a recombinant IgG antibody or antibody fragment comprising an Fc portion mutated to alter (eliminate or enhance) FcR interactions, to increase half-life and/or increase therapeutic efficacy, such as a LALA, LALA PG, N297, GASD/ALIE, DHS, YTE or LS mutation or glycan modified to alter (eliminate or enhance) FcR interactions such as enzymatic or chemical addition or removal of glycans or expression in a cell line engineered with a defined glycosylating pattern.

Delivering may comprise antibody or antibody fragment administration, or genetic delivery with an RNA or DNA sequence or vector encoding the antibody or antibody fragment.

In one embodiment, the method comprises preventing ligand-induced stimulation of TSHR in cells of a subject. In a further embodiment, the ligand is a thyroid stimulating autoantibody or TSH. In a further embodiment, the cells are thyroid cells or extra-thyroidal cells. In particular, the extra-thyroid cells are retro-orbital or pre-tibial cells, more particularly retro-orbital cells.

In some embodiments, methods of inhibiting IGF-1 induced receptor autophosphorylation in a cell by at least 95%, 96%, 97%, 98%, or 99% or by 100% are provided, the method comprising contacting the cell with a multispecific antibody as provided for herein, or a pharmaceutical composition comprising the same.

In a further aspect, the present invention provides an antibody molecule of the invention for use in treating a condition or disorder in which aberrant stimulation of TSHR and/or aberrant signalling of IGF-IR occurs.

In a further aspect, the present invention provides the use of a multispecific antibody molecule of the invention in the manufacture of a medicament for treating a condition or disorder in which aberrant stimulation of TSHR and/or aberrant signalling of IGF-IR occurs.

In one embodiment, the disorder is a cancer comprising cancer cells expressing IGF-1R.

In some embodiments, methods of treating or reducing the severity of, thyroid-associated ophthalmopathy (TAO), or a symptom thereof are provided, the methods comprising administering to a subject a multispecific antibody as provided for herein or a pharmaceutical composition comprising the same.

In some embodiments, methods of treating thyroid eye disease in a subject are provided, the methods comprising administering to a subject a multispecific antibody molecule as provided for herein or a pharmaceutical composition comprising the same.

In some embodiments, methods of reducing Clinical Activity Score (CAS) of thyroid-associated ophthalmopathy (TAO) in a subject are provided, the methods comprising administering to a subject an antibody molecule as provided for herein or a pharmaceutical composition comprising the same.

In some embodiments, methods of a) reducing proptosis by at least 2 mm and b) reducing the clinical activity score (CAS) in a subject with thyroid-associated ophthalmopathy (TAO) are provided, the methods comprising administering to a subject a multispecific antibody molecule as provided for herein or a pharmaceutical composition comprising the same.

In some embodiments, methods of treating or reducing the severity of thyroid-associated ophthalmopathy (TAO) in a subject are provided, the methods comprising administering to a subject a multispecific antibody molecule as provided for herein, or a pharmaceutical composition comprising the same, wherein treatment with said antibody molecule (i) reduces proptosis by at least 2 mm in an eye; (ii) is not accompanied by a deterioration of 2 mm or more in the other (or fellow eye); and (iii) reduces the CAS in said subject to either one (1) or zero (0).

In some embodiments, methods of improving the quality of life in a subject with thyroid-associated ophthalmopathy (TAO, also called Graves' Ophthalmopathy/Graves' Orbitopathy) are provided, the methods comprising administering to a subject an antibody molecule as provided for herein, or a pharmaceutical composition comprising the same. In some embodiments, methods of treating or reducing the severity of diplopia in a subject with thyroid-associated ophthalmopathy (TAO) are provided, the methods comprising administering to a subject an antibody molecule as provided for herein, or a pharmaceutical composition comprising the same.

In some embodiments, embodiments are provided for any of the methods provided for herein, wherein the multispecific antibody, or an antigen binding fragment thereof, is administered in a pharmaceutical composition that additionally comprises a pharmaceutically acceptable diluent or excipient or carrier.

In a further aspect, the present invention provides a kit comprising a multispecific antibody molecule of the present invention, or a pharmaceutical composition thereof.

Advantages of the presently disclosed subject matter will become evident to those of ordinary skill in the art after a study of the description, Figures, and non-limiting Examples in this document.

### BRIEF DESCRIPTION OF THE DRAWINGS

FIG. 1: Representation of an exemplary bispecific antibody targeting TSHR and IGF-1R.

FIG. 2: FACS data for the exemplary bispecific antibody of Example 1.

FIG. 3: LigandTracer Live Cell Kinetics—Cell Binding Traces for an exemplary bispecific antibody of Example 1.

FIG. 4: HA Assay data for an exemplary bispecific antibody of Example 1.

FIG. 5: CAMP Assay data for an exemplary bispecific antibody of Example 1.

### DETAILED DESCRIPTION OF THE INVENTION

The present invention provides multispecific antibodies against insulin like growth factor I receptor (IGF-1R) and thyroid stimulating hormone (TSH) receptor (TSHR), methods for their production, pharmaceutical compositions containing the multispecific antibodies, and uses thereof. More particularly, the presently disclosed invention provides bispecific IGF-1R and TSHR antibodies, and antigen binding fragments thereof. Certain bispecific antibodies and antigen-binding fragments inhibit IGF-1R function or block the biological functions of IGF-1 mediated IGF-1R signalling and inhibit TSHR function or block the biological functions of TSH mediated TSHR signalling.

In certain embodiments, the disclosure relates to multispecific antibodies that inhibit the cellular functions of the IGF-1 receptor. In certain embodiments, the disclosure relates to multispecific antibodies that inhibit binding of IGF-1 and IGF-II to IGF-1R.

In certain embodiments, the disclosure relates to multispecific antibodies that inhibit the cellular functions of the TSHR receptor. In certain embodiments, the disclosure relates to multispecific antibodies which inhibit binding of TSH to TSHR. In certain embodiments, the disclosure relates to multispecific anti-TSH receptor antibodies that antagonize the effects of TSH and which are substantially devoid of agonist activity.

Additionally, the disclosure provides methods for treating thyroid-associated ophthalmopathy (TAO), also known as thyroid eye disease (TED), Graves' ophthalmopathy or orbitopathy (GO), thyrotoxic exophthalmos, dysthyroid ophthalmopathy, and other thyroid eye disorders associated with multispecific antibodies against IGF-1R and TSHR.

As used herein, the terms “antibody” and “antibodies” include full-length antibodies, antigen binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, common light chain antibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. As used herein, the terms “VH” and “VL” refer to antibody heavy and light chain variable domain, respectively, as described in Kabat et al., (1991) Sequences of Proteins of Immunological Interest (NIH Publication No. 91-3242, Bethesda), which is herein incorporated by reference in its entirety.

As used herein, the term “antigen binding protein” or “binding domain” or “binding specificity” refers to a molecule that specifically binds to an antigen as such binding is understood by one skilled in the art. For example, an antigen binding protein that specifically binds to an antigen may bind to other molecules, generally with lower affinity as determined by, e.g., immunoassays (e.g., ELISA), BIAcore®, KinExA 3000 instrument (Sapidyne Instruments, Boise, ID), surface plasmon resonance (SPR) analysis, biolayer interferometry (BLI), or other assays known in the art. In certain embodiments, an antigen-binding moiety that specifically binds to an antigen binds to the antigen with a Ka that is at least 2 logs (e.g., factors of 10), 2.5 logs, 3 logs, 4 logs or greater than the Ka when the molecule binds non-specifically to another antigen.

As used herein, the term “VH/VL pair” refers to a combination of a VH and a VL that together form the binding site for an antigen.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “full-length antibody heavy chain” refers to an antibody heavy chain comprising, from N to C terminal, a VH, a CH1 region, a hinge region, a CH2 domain and a CH3 domain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain. As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th ed. (1991)). Thus, the VHs may be comprised within the corresponding CDRs and references herein to the “hypervariable loops” of VH and VL domains should be interpreted as also encompassing the corresponding CDRs, and vice versa, unless otherwise indicated. “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M. P. et al., “IMGT unique numbering for immunoglobulin and T cell receptor variable domains and Ig superfamily V-like domains,” Dev. Comp. Immunol., 2003 January; 27 (1): 55-77 (“IMGT” numbering scheme), and Honegger A. and Pluckthun A., “Yet another numbering scheme for immunoglobulin variable domains: an automatic modeling and analysis tool,” J. Mol. Biol., 2001 Jun. 8; 309 (3): 657-70, (Aho numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

As used herein, the term “single chain variable fragment” (scFv) refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “human antibody,” as used herein, is intended to include antibodies having variable and Fc domains derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “multispecific antigen-binding molecules,” or “multispecific antibody” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. In certain embodiment, the multispecific antigen binding molecules of the disclosure comprises at least a first binding specificity for a subunit of a receptor and at least a second binding specificity for another receptor subunit. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody of the present disclosure is functionally linked to another antibody or antigen-binding fragment thereof to produce a bispecific antibody with a second binding specificity.

In exemplary embodiments, the heteromeric antibodies of the present disclosure are bispecific antibodies. Bispecific antibodies can be monoclonal, e.g., human or humanized, antibodies that have binding specificities for at least two different antigens. In certain embodiments, the bispecific antibodies of the disclosure comprises at least a first binding domain for a receptor subunit and at least a second binding domain for another receptor subunit.

Methods for making bispecific antibodies are well-known. Traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2, and CH3 regions. It may have the first heavy chain Fc domain (CH1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

As used herein, the term “Fc” refers to a polypeptide comprising a CH2 domain and a CH3 domain, wherein the C-terminus of the CH2 domain is linked (directly or indirectly) to the N-terminus of the CH3 domain. The term “Fc polypeptide” includes an antibody heavy chain linked to an antibody light chain by disulfide bonds (e.g., to form a half-antibody).

In certain embodiments, an Fc chain begins in the hinge region just upstream of the papain cleavage site and ends at the C-terminus of the antibody. Accordingly, a complete Fc chain comprises at least a hinge domain, a CH2 domain, and a CH3 domain. In certain embodiments, an Fc chain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc chain (i.e., a hinge domain, a CH2 domain, and a CH3 domain). In certain embodiments, an Fc chain comprises a hinge domain (or portion thereof) fused to a CH3 domain (or portion thereof). In certain embodiments, an Fc chain comprises a CH2 domain (or portion thereof) fused to a CH3 domain (or portion thereof). In certain embodiments, an Fc chain consists of a CH3 domain or portion thereof. In certain embodiments, an Fc chain consists of a hinge domain (or portion thereof) and a CH3 domain (or portion thereof). In certain embodiments, an Fc chain consists of a CH2 domain (or portion thereof) and a CH3 domain. In certain embodiments, an Fc chain consists of a hinge domain (or portion thereof) and a CH2 domain (or portion thereof). In certain embodiments, an Fc chain lacks at least a portion of a CH2 domain (e.g., all or part of a CH2 domain). An Fc chain herein generally refers to a polypeptide comprising all or part of the Fc chain of an immunoglobulin heavy-chain. This includes, but is not limited to, polypeptides comprising the entire CHI, hinge, CH2, and/or CH3 domains as well as fragments of such peptides comprising only, e.g., the hinge, CH2, and CH3 domain. The Fc chain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As with Fc variants and native Fc's, the term Fc chain includes molecules in monomeric or multimeric form, whether digested from whole antibody or produced by other means. In some embodiment, the Fc chain comprises the carboxy-terminal portions of both heavy chains held together by disulfides. In certain embodiments, an Fc chain consists of a CH2 domain and a CH3 domain.

In some embodiments, an Fc polypeptide comprises part or all of a wild-type hinge sequence (generally at its N-terminal). In some embodiments, an Fc polypeptide does not comprise a functional or wild-type hinge sequence.

As used herein, the term “CH1 domain” refers to the first constant domain of an antibody heavy chain (e.g., amino acid positions 118-215 of human IgG1, according to the EU index). The term includes naturally occurring CH1 domains and engineered variants of naturally occurring CH1 domains (e.g., CH1 domains comprising one or more amino acid insertions, deletions, substitutions, or modifications relative to a naturally occurring CH1 domain).

As used herein, the term “CH2 domain” refers to the second constant domain of an antibody heavy chain (e.g., amino acid positions 231-340 of human IgG1, according to the EU index). The term includes naturally occurring CH2 domains and engineered variants of naturally occurring CH2 domains (e.g., CH2 domains comprising one or more amino acid insertions, deletions, substitutions, or modifications relative to a naturally occurring CH2 domain).

As used herein, the term “CH3 domain” refers to the third constant domain of an antibody heavy chain (e.g., amino acid positions 341-447 of human IgG1, according to the EU index). The term includes naturally occurring CH3 domains and engineered variants of naturally occurring CH3 domains (e.g., CH3 domains comprising one or more amino acid insertions, deletions, substitutions, or modifications relative to a naturally occurring CH3 domain).

As used herein, the term “EU index” refers to the EU numbering convention for the Fc domains of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. All numbering of amino acid positions of the Fc polypeptides, or fragments thereof, used herein is according to the EU index.

As used herein, the term “specifically binds,” “specifically binding,” “binding specificity” or “specifically recognized” refers that an antigen binding protein or antigen-binding fragment thereof that exhibits appreciable affinity for an antigen (e.g., a TSHR antigen, e.g., thyroid stimulating hormone (TSH) and IGF-1R) and does not exhibit significant cross reactivity to a different target protein. As used herein, the term “affinity” refers to the strength of the interaction between an antigen binding protein or antigen-binding fragment thereof antigen binding site and the epitope to which it binds. Methods to determine such specific binding are also well known in the art. In certain embodiments, the antigen binding protein or antigen binding fragment thereof can bind to a human thyroid stimulating hormone receptor (TSHR) and IGF-1R, but not to TSHR and IGF-1R from other species. Alternatively, in some embodiments, the antigen binding proteins or antigen binding fragments bind to human TSHR and IGF-1R and to TSHR and IGF-1R from one or more non-human species. In certain exemplary embodiments, affinity is measured by surface plasmon resonance (SPR), e.g., in a Biacore instrument. As readily understood by those skilled in the art, an antigen binding protein affinity may be reported as a dissociation constant (KD) in molarity (M). The antigen binding protein or antigen-binding fragment thereof of the disclosure have KD values in the range of about 10-5 M to about 10-12 M (i.e., low micromolar to picomolar range), about 10-7 M to 10-11 M, about 10-8 M to about 10-10 M, about 10-9 M. In certain embodiments, the antigen binding protein or antigen-binding fragment thereof has a binding affinity of about 10-5 M, 10-6 M, 10-7 M, 10-8 M, 10-9 M, 10-10 M, 10-11 M, or 10-12 M. In certain embodiments, the antigen binding protein or antigen-binding fragment thereof has a binding affinity of about 10-7 M to about 10-9 M (nanomolar range).

Specific binding can be determined according to any art-recognized means for determining such binding. In some embodiments, specific binding is determined by competitive binding assays (e.g., ELISA) or Biacore assays. In certain embodiments, the assay is conducted at about 20° C., 25° C., 30° C., or 37° C. In certain embodiments, the assay is conducted at physiological pH, at an acidic pH (e.g., a pH more acidic than physiological pH), or at a basic pH (e.g., a pH more basic than physiological pH).

As used herein, “administer” or “administration” refers to the act of injecting or otherwise physically delivering a substance as it exists outside the body (e.g., an isolated binding polypeptide provided herein) into a patient, such as by, but not limited to, subcutaneous, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, mice, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “about” or “approximately” means within about 20%, such as within about 10%, within about 5%, or within about 1% or less of a given value or range.

### TSHR

In an aspect of the present disclosure, provided herein are multispecific antigen binding proteins or antigen binding fragments thereof that bind specifically to thyroid stimulating hormone receptor (TSHR) and IGF-1R. In some embodiments, the TSHR binding proteins block the binding of TSHR autoantibodies to TSHR.

Thyroid function is regulated by TSH secreted by the pituitary (Szkudlinski M W, et al 2002. Physiological Reviews 82:473-502). TSH binds to the TSHR on the surface of thyrocytes and this is the first step in initiating the TSHR signalling cascade. Binding of TSH to the TSHR leads to stimulation of formation and release of thyroid hormones; thyroxine (T4) and tri-iodothyronine (T3). A feedback mechanism involving the levels of T4 and T3 in the circulation and thyrotropin releasing hormone (TRH) secreted by the hypothalamus controls the release of TSH that in turn controls thyroid stimulation and the levels of thyroid hormones in serum (Szkudlinski M W, et al, 2002 supra). The TSHR is a G-protein coupled receptor and is composed of three domains: —a leucine rich repeat domain (LRD), a cleavage domain (CD) and a transmembrane domain (TMD) (Nunez Miguel R, et al 2004. Thyroid 14:991-1011).

Some patients with autoimmune thyroid disease (AITD) develop autoantibodies which are reactive with the TSHR (Rees Smith B, et al 1988. Endocrine Reviews 9:106-121). There are two main types of TSHR autoantibodies (TRAbs); a stimulating type and a blocking type. Thyroid stimulating type autoantibodies bind to the TSHR and mimic the actions of TSH thereby stimulating the thyroid to produce high levels of T4 and T3; these autoantibodies are also described as TRAbs with stimulating activity or TSH agonist activity (Rees Smith B, et al 2007. Thyroid 17:923-938). The feedback control mechanism of thyroid function is no longer effective in the presence of thyroid stimulating autoantibodies and patients present with the clinical symptoms of a hyperactive thyroid characterised by an excess of thyroid hormones in serum and its metabolic consequences. This condition is known as Graves' disease. TRAbs with stimulating activity may also interact with the TSHRs in retroorbital tissue and contribute to the development of the eye signs of Graves' disease. A human monoclonal autoantibody which acts as a powerful thyroid stimulator (hMAb TSHR1; also referred to as M22) has been described in detail in WO2004/050708A2. The structure of the complex of M22 Fab bound to the TSHR LRD has been solved by x-ray crystallography at 2.55 A resolution as described in WO2008/025991A1. Analysis of the structure of the TSHR-M22 complex provides detailed information about the receptor residues and the stimulating autoantibody residues involved in interactions with each other.

Blocking type TRAbs occur less frequently in patients with AITD than stimulating autoantibodies. Blocking type autoantibodies bind to the TSHR, prevent TSH from binding to the receptor but have no ability to stimulate TSHR activity. Consequently formation and secretion of thyroid hormones (T4 and T3) is greatly reduced and the patients with this type of TRAb can present with clinical symptoms of an under-active thyroid (hypothyroidism). Blocking type autoantibodies are known as TRAbs with blocking activity or TSH antagonist activity (Rees Smith B, et al 1988 supra and Rees Smith B, 2007 et al supra). TRAbs with blocking activity when present in serum of pregnant women cross the placenta and may block the TSHRs in the foetal thyroid leading to neonatal hypothyroidism and serious consequences for development. Furthermore, TRAbs with blocking activity can be found in breast milk of affected mothers and may cause clinical hypothyroidism in the baby (Evans C, et al 2004 European Journal of Endocrinology 150:265-268). A human autoantibody to the TSHR with TSH antagonist activity (5C9) has been described in detail in WO 2008/099185A1. Clinical symptoms in patients with AITD and circulating TRAbs are related to the effect of autoantibodies on TSHR activity i.e. whether the TRAbs cause stimulation or blocking. It has been proposed, however, that in some patients a mixture of stimulating and blocking TRAbs may be present simultaneously with the overall clinical presentation related to higher concentration and/or activity of one type of the TRAbs (Rees Smith B et al 1988 supra; Furmaniak J et al 1993 Springer Seminars in Immunopathology 14:309-321 and Schott M et al 2005 Trends in Endocrinology and Metabolism 16:243-248). Furthermore, the concentrations and/or activities of stimulating or blocking TRAb may vary in the same patient during the course of the disease and indeed fluctuation of symptoms from hypo- to hyperthyroidism in the same patient over time has been reported (Rees Smith B et al 1988 supra; Furmaniak J and Rees Smith B 1993 supra and Schott M et al 2005 supra). However, attempts to separate the TRAbs with different bioactivity or to differentiate between these TRAbs in serum samples using currently available bioassays is difficult. More recently, the invention described in WO2006/016121A1 provides a means to discriminate between stimulating and blocking types of TRAbs using bioassays that employ TSHR mutated at R255.

Further TSHR antibodies that may be incorporated into the multispecific antibodies described herein are described in WO2010/073012.

Thyroid-associated ophthalmopathy (TAO), also known as thyroid eye disease (TED), Graves' ophthalmopathy or orbitopathy (GO), thyrotoxic exophthalmos, dysthyroid ophthalmopathy, and several other terms, is orbitopathy associated with thyroid dysfunction. TAO is divided into two types. Active TAO, which typically lasts 1-3 years, is characterized by an ongoing autoimmune/inflammatory response in the soft tissues of the orbit. Active TAO is responsible for the expansion and remodelling of the ocular soft tissues. The autoimmune/inflammatory response of active TAO spontaneously resolves and the condition transitions into inactive TAO. Inactive TAO is the term used to describe the long-term/permanent sequelae of active TAO. The cause of TAO is unknown. TAO is typically associated with Graves' hyperthyroidism, but can also occur as part of other autoimmune conditions that affect the thyroid gland and produce pathology in orbital and periorbital tissue, and, rarely, the pretibial skin (pretibial myxedema) or digits (thyroid acropachy). TAO is an autoimmune orbitopathy in which the orbital and periocular soft tissues are primarily affected with secondary effects on the eye and vision. In TAO, as a result of inflammation and expansion of orbital soft tissues, primarily eye muscles and adipose, the eyes are forced forward (bulge) out of their sockets—a phenomenon termed proptosis or exophthalmos. Although most cases of TAO do not result in loss of vision, this condition can cause vision-threatening exposure keratopathy, troublesome diplopia (double vision), and compressive dysthyroid optic neuropathy. TAO may precede, coincide with, or follow the systemic complications of dysthyroidism. The ocular manifestations of TAO include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences. Many of the signs and symptoms of TAO, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibres, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-Ibeta, interferon-gamma, platelet-derived growth factor, thyroid stimulating hormone (TSH) and insulin-like growth factor I (IGF-I).

### IGF-1R

In an aspect of the present disclosure, provided herein are multispecific antigen binding proteins or antigen binding fragments thereof that bind specifically to Insulin-like growth factor-1 receptor (IGF-1 receptor) and TSHR.

Insulin-like growth factor-1 receptor (IGF-1 receptor) is a transmembrane heterotetrameric protein, which has two extracellular alpha chains and two membrane-spanning beta chains in a disulfide-linked β-α-α-β configuration. The binding of the ligands, which are insulin-like growth-factor-1 (IGF-1) and insulin-like growth factor-II (IGF-II), by the extracellular domain of IGF-1 receptor activates its intracellular tyrosine kinase domain resulting in autophosphorylation of the receptor and substrate phosphorylation. The IGF-1 receptor is homologous to insulin receptor, having a high sequence similarity of 84% in the beta chain tyrosine kinase domain and a low sequence similarity of 48% in the alpha chain extracellular cysteine rich domain (Ulrich, A. et al., 1986, EMBO, 5, 2503-2512: Fujita-Yamaguchi, Y. et al., 1986, J. Biol. Chem., 261, 16727-16731; LeRoith, D. et al., 1995, Endocrine Reviews, 16, 143-163). The IGF-I receptor and its ligands (IGF-I and IGF-II) play important roles in numerous physiological processes including growth and development during embryogenesis, metabolism, cellular proliferation and cell differentiation in adults (LeRoith, D.2000, Endocrinology, 141, 1287-1288; LeRoith, D., 1997, New England J. Med., 336, 633-640). IGF-I and IGF-II function both as endocrine hormones in the blood, where they are predominantly present in complexes with IGF-binding proteins, and as paracrine and autocrine growth factors that are produced locally (Humbel, R. E., 1990, Eur: J. Biochem., 190, 445-462; Cohick, W. S. and Clemmons, D. R., 1993, Annu. Rev. Physiol. 55, 131-153).

The IGF-I receptor has been implicated in promoting growth, transformation and Survival of tumour cells (Baserga, R. et al., 1997, Biochem. Biophys. Acta, 1332, F 105-F126; Blakesley, V. A. et al., 1997, Journal of Endocrinology, 152, 339-344; Kaleko, M., Rutter, W. J., and Miller, A. D. 1990, Mol. Cell. Biol., 10, 464-473). Thus, several types of tumours are known to express higher than normal levels of IGF-I receptor, including breast cancer, colon cancer, ovarian carcinoma, synovial sarcoma and pancreatic cancer (Khandwala, H. M. et al., 2000, Endocrine Reviews, 21, 215-244; Werner, H. and LeRoith, D., 1996, Adv. Cancer Res., 68183-223; Happerfield, L. C. et al., 1997, J. Pathol., 183, 412-417; Frier, S. et al., 1999, Gut, 44, 704-708; van Dam, P. A. et al., 1994, J. Clin. Pathol., 47,914-919; Xie, Y. et al., 1999, Cancer Res., 59,3588-3591; Bergmann, U. et al., 1995, Cancer Res., 55, 2007-2011). In vitro, IGF-I and IGF-II have been shown to be potent mitogens for several human tumour cell lines such as lung cancer, breast cancer, colon cancer, osteosarcoma and cervical cancer (Ankrapp, D. P. and Bevan, mD. R., 1993, Cancer Res., 53,3399-3404; Cullen, K. J., 1990, Cancer Res., 50, 48-53: Hermanto, U. et al., 2000, Cell Growth & Differentiation, 11,655-664: Guo, Y. S. et al., 1995, J. Am. Coll. Surg., 181, 145-154; Kappel, C. C. et al., 1994, Cancer Res., 54, 2803-2807; Steller, M. A. et al., 1996, Cancer Res., 56, 1761-1765). Several of these tumours and tumour cell lines also express high levels of IGF-I or IGF-II, which may stimulate their growth in an autocrine or paracrine manner (Quinn, K. A. et al., 1996, J. Biol. Chem., 271, 11477-11483).

### Effector Function Mutations

The antigen binding proteins of the disclosure (e.g., the multispecific antibodies targeting TSHR and IGF-1R) can be provided in various isotypes and with different Fc domains. The Fc region of the antigen binding protein primarily determines its effector function in terms of Fc binding, antibody-dependent cell-mediated cytotoxicity (ADCC) activity, complement dependent cytotoxicity (CDC) activity, and antibody-dependent cell phagocytosis (ADCP) activity. These “cellular effector functions”, as distinct from effector T cell function, involve the recruitment of cells bearing Fc receptors to the site of the target cells, resulting in killing of the antibody-bound cell.

An antigen binding protein or an antigen binding fragment thereof according to the present invention may be one that exhibits reduced effector function. In certain embodiments, the one or more mutations reduces one or more of antibody dependent cellular cytotoxicity (ADCC), antibody dependent cellular phagocytosis (ADCP), or complement dependent cytotoxicity (CDC). In certain embodiments, an antibody according to the present invention may lack ADCC, ADCP, and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

In certain embodiments, the first and the second Fc domain comprises one or more mutations that reduces Fc effector function. In certain embodiments, the first Fc domain and the second Fc domain each comprise a L234A and L235A mutation. These IgG1 mutations are also known as the “LALA” mutations and are described in further detail in Xu et al. (Cell Immunol. 2000; 200:16-26). In certain embodiments, the first Fc domain and the second Fc domain each comprise a L234A, L235A, G237A, and/or P329G mutations. The Fc domain amino acid positions referred to herein are based on EU antibody numbering. Alternatively, an antibody may have a Fc domain which is effector null. An antibody may have a heavy chain Fc domain that does not bind Fcγ receptors, for example the Fc domain may comprise a L235E mutation. Another optional mutation for a heavy chain Fc domain is S228P, which increases stability. A heavy chain Fc domain may be an IgG4 comprising both the L235E mutation and the S228P mutation. This “IgG4-PE” heavy chain Fc domain is effector null. A disabled IgG1 heavy chain Fc domain may contain alanine at position 234, 235, and/or 237 (EU index numbering), e.g., it may be an IgG1 sequence comprising the L234A, L235A, and/or G237A mutations (“LALAGA”).

Human IgG1 Fc domains containing specific mutations or altered glycosylation on residue Asn297 (e.g., N297Q, N297D, and N297K, according to EU index numbering) have been shown to reduce binding to Fc receptors.

In other embodiments, it may be desirable to enhance the binding of the Fc region of an antigen binding protein to human Fc gamma receptor IIIA (FcγRIIIA) relative to that of the Fc region of a corresponding naturally occurring antibody. In certain embodiments, a Fc domain may be engineered for enhanced ADCC and/or CDC and/or ADCP. The potency of Fc-mediated effects may be enhanced by engineering the Fc domain by various established techniques. Such methods increase the affinity for certain Fc-receptors, thus creating potential diverse profiles of activation enhancement. This can be achieved by modification of one or several amino acid residues. Example mutations are one or more of the residues selected from 239, 332 and 330 for human IgG1 Fc domains (or the equivalent positions in other IgG isotypes). An antibody may thus comprise a human IgG1 Fc domain having one or more mutations independently selected from S239D, I332E and A330L (EU index numbering).

Increased affinity for Fc receptors can also be achieved by altering the natural glycosylation profile of the Fc domain by, for example, generating under fucosylated or de-fucosylated variants. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIA binding capacity. For example, to increase ADCC, residues in the hinge region can be altered to increase binding to FcγRIIIA. Thus, an antibody may comprise a human IgG heavy chain Fc domain that is a variant of a wild-type human IgG heavy chain Fc domain. In certain embodiments, the variant human IgG heavy chain Fc domain binds to human Fcγ receptors selected from the group consisting of FcγRIIB and FcγRILA with higher affinity than the wild type human IgG heavy chain Fc domain binds to the human FcγRIIIA. The antibody may comprise a human IgG heavy chain Fc domain that is a variant of a wild type human IgG heavy chain Fc domain, wherein the variant human IgG heavy chain Fc domain binds to human FcγRIIB with higher affinity than the wild type human IgG heavy chain Fc domain binds to human FcγRIIB. The variant human IgG heavy chain Fc domain can be a variant human IgG1, a variant human IgG2, or a variant human IgG4 heavy chain Fc domain. In one embodiment, the variant human IgG heavy chain Fc domain comprises one or more amino acid mutations selected from G236D, P238D, S239D, S267E, L328F, and L328E (EU index numbering system). in another embodiment, the variant human IgG heavy chain Fc domain comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system).

The enhancement of CDC may be achieved by amino acid changes that increase affinity for C1q, the first component of the classic complement activation cascade. Another approach is to create a chimeric Fc domain created from human IgG1 and human IgG3 segments that exploit the higher affinity of IgG3 for C1q. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a Fc domain comprising one or more mutations selected from E345K, E430G, R344D and D356R, in particular a double mutation comprising R344D and D356R (EU index numbering system).

The functional properties of the antigen binding proteins may be further tuned by combining amino acid substitutions that alter Fc binding affinity with amino acid substitutions that affect binding to FcRn. Binding proteins with amino acid substitutions that affect binding to FcRn (also referred to herein as “FcRn variants”) may in certain situations also increase serum half-life in vivo as compared to an unmodified binding protein. As it will be appreciated, any combination of Fc and FcRn variants may be used to tune clearance of the antigen-antibody complex. Suitable FcRn variants that may be combined with any of the Fc variants described herein that include without limitation N434A, N434S, M428L, V308F, V2591, M428L/N434S, V2591/V308F, Y4361/M428L, Y4361/N434S, Y436V/N434S, Y436V/M428L, M252Y, M252Y/S254T/T256E, and V2591/V308F/M428L.

### Heterodimerization Motifs

In certain exemplary embodiments, the first and second Fc domains of the TSHR x IGF-1R multispecific antibodies as disclosed herein are further engineered to enhance heterodimerization of the first specific and second specific binding domains and minimize the effects of incorrect chain pairing.

Any art-recognized approach that addresses the problem of incorrect chain pairing can be employed to improve desired antigen binding protein production. For example, for bispecific antibodies, US2010/0254989 A1 describes the construction of bispecific cMet—ErbB1 antibodies, where the VH and VL of the individual antibodies are fused genetically via a GlySer linker. For bispecific antibodies including an Fc domain, mutations may be introduced into the Fc to promote the correct heterodimerization of the Fc portion. Several such approaches are reviewed in Klein et al. (mAbs (2012) 4:6, 1-11), the contents of which are incorporated herein by reference in their entirety.

In certain embodiments, the binding specificities of a multispecific antibody are heterodimerized through knobs-into-holes (KiH) pairing of Fc domains. This dimerization technique utilizes “protuberances” or “knobs” with “cavities” or “holes” engineered into the interface of CH3 domains. Where a suitably positioned and dimensioned knob or hole exists at the interface of either the first or second CH3 domain, it is only necessary to engineer a corresponding hole or knob, respectively, at the adjacent interface, thus promoting and strengthening Fc domain pairing in the CH3/CH3 domain interface. The IgG Fc domain that is fused to the binding region is provided with a knob, and the IgG Fc domain of the conventional antibody is provided with a hole designed to accommodate the knob, or vice-versa. A “knob” refers to an at least one amino acid side chain, typically a larger side chain, that protrudes from the interface of the CH3 portion of a first Fc domain. The protrusion creates a “knob” which is complementary to and received by a “hole” in the CH3 portion of a second Fc domain. The “hole” is an at least one amino acid side chain, typically a smaller side chain, which recedes from the interface of the CH3 portion of the second Fc domain. This technology is described, for example, in U.S. Pat. Nos. 5,821,333; 5,731,168 and 8,216,805; Ridgway et al. Protein Engineering (1996) 9:617-621); and Carter P. J. Immunol. Methods (2001) 248:7-15, which are herein incorporated by reference.

Exemplary amino acid residues that may act as the knob include arginine (R), phenylalanine (F), tyrosine (Y) or tryptophan (W). An existing amino acid residue in the CH3 domain may be replaced or substituted with a knob amino acid residue. Preferred amino acids to substitute may include any amino acids with a small side chain, such as alanine (A), asparagine (N), aspartic acid (D), glycine (G), serine(S), threonine (T), or valine (V).

Exemplary amino acid residues that may act as the hole include alanine (A), serine (S), threonine (T), or valine (V). An existing amino acid residue in the CH3 domain may be replaced or substituted with a hole amino acid residue. Preferred amino acids to substitute may include any amino acids with a large side chain, such as arginine (R), phenylalanine (F), tyrosine (Y) or tryptophan (W). The CH3 domain is preferably derived from a human IgG1 antibody. Exemplary amino acid substitutions to the CH3 domain include Y349C, S354C, T366S, T366Y, T366W, F405A, F405W, Y407T, Y407A, Y407V, T394S, or combinations thereof. A preferred exemplary combination is S354C, T366Y or T366W for the knob mutation on a first CH3 domain and Y349C, T366S, L368A, Y407T or Y407V for the hole mutation on a second CH3 domain.

In certain embodiments, the two Fc domains of the antigen binding construct are heterodimerized through Fab arm exchange (FAE). A human IgG1 possessing a P228S hinge mutation may contain an F405L or K409R CH3 domain mutation. Mixing of the two antibodies with a reducing agent leads to FAE. This technology is described in U.S. Pat. No. 9,212,230 and Labrijn A. F. PNAS (2013) 110 (13): 5145-5150, which are incorporated herein by reference.

In other embodiments, the two Fc domains of the antigen binding construct are heterodimerized through electrostatic steering effects. This dimerization technique utilizes electrostatic steering to promote and strengthen Fc domain pairing in the CH3/CH3 domain interface. The charge complementarity between two CH3 domains is altered to favor heterodimerization (opposite charge paring) over homodimerization (same charge pairing). In this method, the electrostatic repulsive forces prevent homodimerization. Certain exemplary amino acid residue substitutions which confer electrostatic steering effects include K409D, K392D, and/or K370D in a first CH3 domain and D399K, E356K, and/or E357K in a second CH3 domain. This technology is described in US Patent Publication No. 2014/0154254 A1 and Gunasekaran K. JBC (2010) 285 (25): 19637-19646, which are incorporated herein by reference.

In other embodiments, the charge complementarity is formed by a first Fc domain comprising a N297K and/or a T299K mutation, and a second Fc domain comprising a N297D and/or a T299D mutation.

In certain embodiments, the two Fc domains of the antigen binding construct are heterodimerized through hydrophobic interaction effects. This dimerization technique utilizes hydrophobic interactions instead of electrostatic ones to promote and strengthen Fc domain pairing in the CH3/CH3 domain interface. Exemplary amino acid residue substitution may include K409W, K360E, Q347E, Y349S, and/or S354C in a first CH3 domain and D399V, F405T, Q347R, E357W, and/or Y349C in a second CH3 domain. Preferred pairs of amino acid residue substitutions between a first CH3 domain and a second CH3 domain include K409W:D399V, K409W:F405T, K360E:Q347R, Y349S:E357W, and S354C:Y349C. This technology is described in US Patent Publication No. 2015/0307628 A1.

In certain embodiments, heterodimerization can be mediated through the use of leucine zipper fusions. Leucine zipper domains fused to the C terminus of each CH3 domain of the antibody chains force heterodimerization. This technology is described in Wranik B. JBC (2012) 287 (52): 43331-43339.

In certain embodiments, heterodimerization can be mediated through the use of a Strand Exchange Engineered Domain (SEED) body. CH3 domains derived from an IgG and IgA format force heterodimerization. This technology is described in Muda M. PEDS (2011) 24 (5): 447-454.

In certain embodiments, the heterodimerization motif may comprise non-native, disulfide bonds formed by engineered cysteine residues. In certain embodiments, the first set of disulfide may comprise a Y349C mutation in the first Fc domain and a S354C mutation in the second Fc domain. In other embodiment, an engineered disulfide bond may be introduced by fusion a C-terminal extension peptide with an engineered cysteine residue to the C-terminus of each of the two Fc domains. In certain embodiments, the first Fc domain may comprise the substitution of the carboxyl-terminal as “PGK” with “GEC”, and the second Fc domain may comprise the substitution of the carboxyl terminal amino acids “PGK” with “KSCDKT” (SEQ ID NO: 140).

In certain embodiments, the antigen binding proteins may employ the CrossMab principle (as reviewed in Klein et al.), which involves domain swapping between heavy and light chains so as to promote the formation of the correct pairings. Yet another approach involves engineering the interfaces between the paired VH-VL domains or paired CH1-CL domains of the heavy and light chains to increase the affinity between the heavy chain and its cognate light chain (Lewis et al. Nature Biotechnology (2014) 32:191-198).

An alternative approach to the production of an antigen binding protein preparations having the correct antigen specificity has been the development of methods that enrich for antibodies having the correct heavy chain-light chain pairings. For example, Spiess et al. (Nature Biotechnology (2013) 31:753-758) describe a method for the production of a MET-EGFR bispecific antibody from a co-culture of bacteria expressing two distinct half-antibodies.

Methods have also been described wherein the Fc domain of at least one of the heavy chains of a bispecific antibody is mutated so as to alter its binding affinity for an affinity agent, for example Protein A. This allows correctly paired heavy chain heterodimers to be isolated based on a purification technique that exploits the differential binding of the two heavy chains to an affinity agent (see US2010/0331527, WO2013/136186).

International patent application no. PCT/EP2012/071866 (WO2013/064701) addresses the problem of incorrect chain pairing using a method for multispecific antibody isolation based on the use of anti-idiotypic binding agents, in particular anti-idiotypic antibodies. The anti-idiotype binding agents are employed in a two-step selection method in which a first agent is used to capture antibodies having a VH-VL domain pairing specific for a first antigen and a second agent is subsequently used to capture antibodies also having a second VH-VL domain pairing specific for a second antigen.

In certain other embodiments, the antigen binding protein described herein further comprises a common light chain. The term “common light chain” as used herein refers to a light chain which is capable of pairing with a first heavy chain of an antibody which binds to a first antigen in order to form a binding site specifically binding to said first antigen and which is also capable of pairing with a second heavy chain of an antibody which binds to a second antigen in order to form a binding site specifically binding to said second antigen. A common light chain is a polypeptide comprising in N-terminal to C-terminal direction an antibody light chain variable domain (VL), and an antibody light chain constant domain (CL), which is herein also abbreviated as “VL-CL”. Multispecific binding proteins with a common light chain require heterodimerization of the distinct heavy chains. In certain embodiments, the heterodimerization methods listed above may be used with a common light chain. In certain exemplary embodiments, the heterodimerization motif may comprise non-native, disulfide bonds formed by engineered cysteine residues. Adding disulfide bonds, both between the heavy and light chain of an antibody has been shown to improve stability. Additionally, disulfide bonds have also been used as a solution to improve light-chain pairing within bispecific antibodies (Geddie M. L. et al, mABs (2022) 14 (1)).

Unless otherwise stated, all antibody Fc domain numbering employed herein corresponds to the EU numbering scheme, as described in Edelman et al. (Proc. Natl. Acad. Sci. 63 (1): 78-85. 1969).

Additional methods of heterodimerization of heavy and/or light chains and the generation and purification of asymmetric antibodies are known in the art. See, for example, Klein C. mAbs (2012) 4 (6): 653-663, and U.S. Pat. No. 9,499,634, each of which is incorporated herein by reference.

The present disclosure also contemplates isotype modification. By modifying the Fc region to have a different isotype, different functionalities can be achieved. For example, changing to IgG1 can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23pl9 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγR III binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγR I, FcγR II, FcγR III, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behaviour. Computational strategies followed by mutagenesis may also be used to select one of amino acid mutations to mutate.

The present disclosure therefore provides a variant of an antigen binding protein with optimized binding to FcRn. In a particular embodiment, the variant of an antigen binding protein comprises at least one amino acid modification in the Fc region of the antigen binding protein, wherein the modification is selected from the group consisting of 226, 227, 228, 230, 231, 233, 234, 239, 241, 243, 246, 250, 252, 254, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401403, 404, 408, 411, 412, 414, 415, 416, 418, 419, 420, 421, 422, 424, 426, 428, 433, 434, 438, 439, 440, 443, 444, 445, 446 and 447 of the Fc region as compared to the parent polypeptide, wherein the numbering of the amino acids in the Fc region is that of the EU index in Kabat. In a further embodiment of the disclosure the modifications are M252Y/S254T/T256E. In a further embodiment of the disclosure the modifications are M252Y/S254T/T256E. In a further embodiment of the disclosure, the modifications are T250Q/M428L

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fe receptors have been greatly reduced or fusing with FcRn binding domains of antibodies.

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably in a human, results in a higher serum titre of the antibodies or antibody fragments in the mammal, and thus reduces the frequency of the administration of the antibodies or antibody fragments and/or reduces the concentration of the antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fe domain and the FcRn receptor. Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγR I, FcγR II and FcγR IIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies. In a further embodiment of the disclosure the modifications are L234A/L235A (LALA) or L234A/L235A+P329G (LALA-PG).

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21 (2), 385-392, 2010) Evidence from solubility-altering mutations in the literature indicate that some hydrophilic residues such as aspartic acid, glutamic acid, and serine contribute significantly more favourably to protein solubility than other hydrophilic residues, such as asparagine, glutamine, threonine, lysine, and arginine.

Elevated temperature may be used to unfold antibodies to determine relative stability, using average apparent melting temperatures. Differential Scanning calorimetry (DSC) measures the heat capacity, Cp, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, CH2, and CH3 domains). Typically unfolding of the Fab domain produces the strongest peak. The DSC profiles and relative stability of the Fe portion show characteristic differences for the human IgG1, IgG2, IgG3, and IgG4 subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not diverse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an ICE approach to resolve antibody protein charge variants due to deamidation, C-terminal lysines, sialylation, oxidation, glycosylation, and any other change to the protein that can result in a change in pH of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pls). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in CDRH3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366:449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Generally, it is thought that autoreactive clones should be eliminated during ontogeny by negative selection, however it has become clear that many human naturally occurring antibodies with autoreactive properties persist in adult mature repertoires, and the autoreactivity may enhance the antiviral function of many antibodies to pathogens. It has been noted that HCDR3 loops in antibodies during early B cell development are often rich in positive charge and exhibit autoreactive patterns (Wardemann et al., Science 301, 1374-1377, 2003).

A given antibody may be tested for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. It is possible to assess antibody characteristics of the combined antibody repertoire of healthy human blood donors of about 400 million sequences in total and create a novel “relative Human Likeness” (rHL) score that focuses on the hypervariable region of the antibody. The rHL score may easily distinguish between human (positive score) and non-human sequences (negative score). Antibodies can be engineered to eliminate residues that are not common in human repertoires.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

Typically, a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido)ethyl-1,3′-dithiopropionate. The N-hydroxy-succinimidy I group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment includes the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a praline, and is characterized by greater stability and reduced aggregation. U.S. Pat. No. 5,880,270 discloses aminooxy-containing linkers useful in a variety of immunodiagnostic and separative techniques.

Artificial T cell receptors (also known as chimeric T cell receptors, chimeric immunoreceptors, chimeric antigen receptors (CARs)) are engineered receptors, which graft an arbitrary specificity onto an immune effector cell. Typically, these receptors are used to graft the specificity of a monoclonal antibody onto a T cell, with transfer of their coding sequence facilitated by retroviral vectors. In this way, a large number of target-specific T cells can be generated for adoptive cell transfer. Phase I clinical studies of this approach show efficacy.

The most common form of these molecules are fusions of single-chain variable fragments (scFv) derived from monoclonal antibodies, fused to CD3-zeta transmembrane and endodomain. Such molecules result in the transmission of a zeta signal in response to recognition by the scFv of its target. An example of such a construct is 14g2a-Zeta, which is a fusion of a scFv derived from hybridoma 14g2a (which recognizes disialoganglioside GD2). When T cells express this molecule (usually achieved by oncoretroviral vector transduction), they recognize and kill target cells that express GD2 (e.g., neuroblastoma cells). To target malignant B cells, investigators have redirected the specificity of T cells using a chimeric immunoreceptor specific for the B-lineage molecule, CD19.

The variable portions of an immunoglobulin heavy and light chain are fused by a flexible linker to form a scFv. This scFv is preceded by a signal peptide to direct the nascent protein to the endoplasmic reticulum and subsequent surface expression (this is cleaved). A flexible spacer allows to the scFv to orient in different directions to enable antigen binding. The transmembrane domain is a typical hydrophobic alpha helix usually derived from the original molecule of the signaling endodomain which protrudes into the cell and transmits the desired signal.

Type I proteins are in fact two protein domains linked by a transmembrane alpha helix in between. The cell membrane lipid bilayer, through which the transmembrane domain passes, acts to isolate the inside portion (endodomain) from the external portion (ectodomain). It is not so surprising that attaching an ectodomain from one protein to an endodomain of another protein results in a molecule that combines the recognition of the former to the signal of the latter.

With regards the ectodomain, a signal peptide directs the nascent protein into the endoplasmic reticulum. This is essential if the receptor is to be glycosylated and anchored in the cell membrane. Any eukaryotic signal peptide sequence usually works fine. Generally, the signal peptide natively attached to the amino-terminal most component is used (e.g., in a scFv with orientation light chain-linker-heavy chain, the native signal of the light-chain is used

The antigen recognition domain is usually an scFv. There are however many alternatives. An antigen recognition domain from native T-cell receptor (TCR) alpha and beta single chains have been described, as have simple ectodomains (e.g., CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

A spacer region links the antigen binding domain to the transmembrane domain. It should be flexible enough to allow the antigen binding domain to orient in different directions to facilitate antigen recognition. The simplest form is the hinge region from IgG1. Alternatives include the CH2CH3 region of immunoglobulin and portions of CD3. For most scFv based constructs, the IgG1 hinge suffices.

The transmembrane domain is a hydrophobic alpha helix that spans the membrane. Generally, the transmembrane domain from the most membrane proximal component of the endodomain is used. Interestingly, using the CD3-zeta transmembrane domain may result in incorporation of the artificial TCR into the native TCR a factor that is dependent on the presence of the native CD3-zeta transmembrane charged aspartic acid residue. Different transmembrane domains result in different receptor stability. The CD28 transmembrane domain results in a brightly expressed, stable receptor.

The endodomain is the “business-end” of the receptor. After antigen recognition, receptors cluster and a signal is transmitted to the cell. The most commonly used endodomain component is CD3-zeta which contains 3 ITAMs. This transmits an activation signal to the T cell after antigen is bound. CD3-zeta may not provide a fully competent activation signal and additional co-stimulatory signaling is needed.

“First-generation” CARs typically had the intracellular domain from the CD3E-chain, which is the primary transmitter of signals from endogenous TCRs. “Second-generation” CARs add intracellular signaling domains from various costimulatory protein receptors (e.g., CD28, 41BB, ICOS) to the cytoplasmic tail of the CAR to provide additional signals to the T cell. Preclinical studies have indicated that the second generation of CAR designs improves the antitumour activity of T cells. More recent, “third-generation” CARs combine multiple signaling domains, such as CD3z-CD28-41BB or CD3z-CD28-OX40, to further augment

Antibody Drug Conjugates or ADCs are a new class of highly potent biopharmaceutical drugs designed as a targeted therapy for the treatment of people with infectious disease. ADCs are complex molecules composed of an antibody (a whole mAb or an antibody fragment such as a single-chain variable fragment, or scFv) linked, via a stable chemical linker with labile bonds, to a biological active cytotoxic/anti-viral payload or drug. Antibody Drug Conjugates are examples of bioconjugates and immunoconjugates.

By combining the unique targeting capabilities of monoclonal antibodies with the cancer-killing ability of cytotoxic drugs, antibody-drug conjugates allow sensitive discrimination between healthy and diseased tissue. This means that, in contrast to traditional systemic approaches, antibody-drug conjugates target and attack the infected cell so that healthy cells are less severely affected.

In the development ADC-based anti-tumour therapies, an anticancer drug (e.g., a cell toxin or cytotoxin) is coupled to an antibody that specifically targets a certain cell marker (e.g., a protein that, ideally, is only to be found in or on infected cells). Antibodies track these proteins down in the body and attach themselves to the surface of cancer cells. The biochemical reaction between the antibody and the target protein (antigen) triggers a signal in the tumour cell, which then absorbs or internalizes the antibody together with the cytotoxin. After the ADC is internalized, the cytotoxic drug is released and kills the cell or impairs viral replication. Due to this targeting, ideally the drug has lower side effects and gives a wider therapeutic window than other agents.

A stable link between the antibody and cytotoxic/anti-viral agent is a crucial aspect of an ADC. Linkers are based on chemical motifs including disulfides, hydrazones or peptides (cleavable), or thioethers (noncleavable) and control the distribution and delivery of the cytotoxic agent to the target cell. Cleavable and noncleavable types of linkers have been proven to be safe in preclinical and clinical trials. Brentuximab vedotin includes an enzyme-sensitive cleavable linker that delivers the potent and highly toxic antimicrotubule agent Monomethyl auristatin E or MMAE, a synthetic antineoplastic agent, to human specific CD30-positive malignant cells. Because of its high toxicity MMAE, which inhibits cell division by blocking the polymerization of tubulin, cannot be used as a single-agent chemotherapeutic drug. However, the combination of MMAE linked to an anti-CD30 monoclonal antibody (cACIO, a cell membrane protein of the tumour necrosis factor or TNF receptor) proved to be stable in extracellular fluid, cleavable by cathepsin and safe for therapy. Trastuzumab emtansine, the other approved ADC, is a combination of the microtubule-formation inhibitor mertansine (DM-1), a derivative of the Maytansine, and antibody trastuzumab (Herceptin®/Genentech/Roche) attached by a stable, non-cleavable linker.

The availability of better and more stable linkers has changed the function of the chemical bond. The type of linker, cleavable or noncleavable, lends specific properties to the cytotoxic (anti-cancer) drug. For example, a non-cleavable linker keeps the drug within the cell. As a result, the entire antibody, linker and cytotoxic agent enter the targeted cancer cell where the antibody is degraded to the level of an amino acid. The resulting complex—amino acid, linker and cytotoxic agent—now becomes the active drug. In contrast, cleavable linkers are catalyzed by enzymes in the host cell where it releases the cytotoxic agent.

Another type of cleavable linker, currently in development, adds an extra molecule between the cytotoxic/anti-viral drug and the cleavage site. This linker technology allows researchers to create ADCs with more flexibility without worrying about changing cleavage kinetics. Researchers are also developing a new method of peptide cleavage based on Edman degradation, a method of sequencing amino acids in a peptide. Future direction in the development of ADCs also include the development of site-specific conjugation (TDCs) to further improve stability and therapeutic index and a emitting immunoconjugates and antibody-conjugated nanoparticles.

Bi-specific T-cell engagers (BiTEs) are a class of artificial bispecific monoclonal antibodies that are investigated for the use as anti-cancer drugs. They direct a host's immune system, more specifically the T cells' cytotoxic activity, against infected cells. BiTE is a registered trademark of Micromet AG.

BiTEs are fusion proteins consisting of two single-chain variable fragments (scFvs) of different antibodies, or amino acid sequences from four different genes, on a single peptide chain of about 55 kilodaltons. One of the scFvs binds to T cells via the CD3 receptor, and the other to an infected cell via a specific molecule.

Like other bispecific antibodies, and unlike ordinary monoclonal antibodies, BiTEs form a link between T cells and target cells. This causes T cells to exert cytotoxic/anti-viral activity on infected cells by producing proteins like perforin and granzymes, independently of the presence of MHC I or co-stimulatory molecules. These proteins enter infected cells and initiate the cell's apoptosis. This action mimics physiological processes observed during T cell attacks against infected cells.

In a particular embodiment, the antibody is a recombinant antibody that is suitable for action inside of a cell—such antibodies are known as “intrabodies.” These antibodies may interfere with target function by a variety of mechanism, such as by altering intracellular protein trafficking, interfering with enzymatic function, and blocking protein-protein or protein-DNA interactions. In many ways, their structures mimic or parallel those of single chain and single domain antibodies, discussed above. Indeed, single-transcript/single-chain is an important feature that permits intracellular expression in a target cell, and also makes protein transit across cell membranes more feasible. However, additional features are required.

The two major issues impacting the implementation of intrabody therapeutic are delivery, including cell/tissue targeting, and stability. With respect to delivery, a variety of approaches have been employed, such as tissue-directed delivery, use of cell-type specific promoters, viral-based delivery and use of cell-permeability/membrane translocating peptides. With respect to the stability, the approach is generally to either screen by brute force, including methods that involve phage display and may include sequence maturation or development of consensus sequences, or more directed modifications such as insertion stabilizing sequences (e.g., Fc regions, chaperone protein sequences, leucine zippers) and disulfide replacement/modification.

An additional feature that intrabodies may require is a signal for intracellular targeting. Vectors that can target intrabodies (or other proteins) to subcellular regions such as the cytoplasm, nucleus, mitochondria and ER have been designed and are commercially available (Invitrogen Corp.; Persic et al., 1997).

By virtue of their ability to enter cells, intrabodies have additional uses that other types of antibodies may not achieve. In the case of the present antibodies, the ability to interact with the MUCI cytoplasmic domain in a living cell may interfere with functions associated with the MUCI CD, such as signalling functions (binding to other molecules) or oligomer formation. In particular, it is contemplated that such antibodies can be used to inhibit MUC1 dimer formation.

The present disclosure provides pharmaceutical compositions comprising anti-IGF-1R×anti-TSHR multispecific antibodies. Such compositions comprise a prophylactically or therapeutically effective amount of the multispecific antibody or a fragment thereof, and a pharmaceutically acceptable carrier. The compositions of the invention may include an “effective amount” or “therapeutically effective amount” or a “prophylactically effective amount” of an antibody or antigen-binding portion of the invention. These terms are used interchangeably. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the subject, and the ability of the antibody or antibody portion to elicit a desired response in the subject. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

“Prevention” of a condition or disorder refers to delaying or preventing the onset of a condition or disorder or reducing its severity, as assessed by the appearance or extent of one or more symptoms of the condition or disorder.

As used herein, the term “subject” refers to an animal. Typically the animal is a mammal. A subject also refers to for example, primates (e.g., humans), cows, sheep, goats, horses, dogs, cats, rabbits, rats, mice, fish, birds and the like. In certain embodiments, the subject is a primate. In yet other embodiments, the subject is a human.

As used herein, a subject is “in need of” a treatment if such subject would benefit biologically, medically or in quality of life from such treatment.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavouring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumour agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules chemiluminescent molecules, chromophores, photoaffinity molecules, coloured particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine211, 14carbon, 51chromium, 36chlorine, 57cobalt, 58cobalt, copper67, 152Eu, gallium67, 3hydrogen, iodine123, iodine125, iodine131, indium111, 59iron, 32phosphorus, rhenium186, rhenium188, 75selenium, 35sulphur, technicium99m and/or yttrium90. 125I is often being preferred for use in certain embodiments, and technicium99m and/or indium111 are also often preferred due to their low energy and suitability for long range detection. Radioactively labelled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labelled with technetium99m by ligand exchange process, for example, by reducing pertechnate with stannous solution, chelating the reduced technetium onto a Sephadex column and applying the antibody to this column. Alternatively, direct labelling techniques may be used, e.g., by incubating pertechnate, a reducing agent such as SnCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a coloured product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3a-6a-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumours is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

TABLE 1A
PROTEIN SEQUENCES FOR TSHR ANTIBODY
VARIABLE REGION

| | SEQ | | |
|---|---|---|---|
| | ID | | |
| Clone | NO | Chain | Variable Sequence Region |
| K1-70 | 1 | heavy | EVQLVQSGAEVKKPGQSLKISCKASGYS |
| | | | LTDNWIGWVRQKPGKGLEWMGIIYPGDS |
| | | | DTRYSPSFQGQVTISADKSINTAYLQWS |
| | | | SLKASDTAIYYCVGLDWNYNPLRYWGPG |
| | | | TLVTVSS |
| | 2 | light | QSVLTQPPSVSAAPGQKVTISCSGSSSD |
| | | | IGSNYVSWYQQFPGTAPKLLIYDNNKRP |
| | | | SAIPDRFSGSKSGTSATLGITGLQTGDE |
| | | | ADYYCGTWDSRLGIAVFGGGTQLTVL |

TABLE 1B
PROTEIN SEQUENCES FOR TSHR ANTIBODY VARIABLE REGION

| | SEQ ID | |
|---|---|---|
| Clone | NO | Variable Sequence Region |
| 1-46 VH | 33 | EVQLVQSGAEVKKPGASVKVSCKASGYSLTDNWIGWVRQAPG |
| | | QGLEWMGIIYPGDSDTRYAQKFQGRVTMTADKSTSTVYMELSS |
| | | LRSEDTAVYYCVGLDWNYNPLRYWGQGTLVTVSS |
| 1-40 VL | 34 | QSVLTQPPSVSGAPGQRVTISCSGSSSDIGSNYVSWYQQLPGT |
| | | APKLLIYDNNKRPSGVPDRFSGSKSGTSASLAITGLQAEDEADY |
| | | YCGTWDSRLGIAVFGGGTKLTVL |
| GL_ 5- | 35 | EVQLVQSGAEVKKPGESLKISCKASGYELNDNWIGWVRQKPG |
| 51_VH1 | | KGLEWMGIIYPGDSDTRYSPSFKGQVTISADKSISTAYLQWSSL |
| | | KASDTAIYYCVGLEWNYNPLRYWGQGTLVTVSS |
| GL_5-51 | 36 | EVQLVQSGAEVKKPGESLKISCKASGYQLNDNWIGWVRQKPG |
| VH2 | | KGLEWMGIIYPGDSDTRYSPSFKGQVTISADKSISTAYLQWSSL |
| | | KASDTAIYYCVGLEWNYNPLRYWGQGTLVTVSS |
| GL_5-51 | 37 | EVQLVQSGAEVKKPGESLKISCKASGYELKDNWIGWVRQKPGK |
| VH3 | | GLEWMGIIYPGDSDTRYSPSFKGQVTISADKSISTAYLQWSSLK |
| | | ASDTAIYYCVGLEWNYNPLRYWGQGTLVTVSS |
| GL_5-51 | 38 | EVQLVQSGAEVKKPGESLKISCKASGYQLKDNWAGWVRQKPG |
| VH4 | | KGLEWMGIIYPGDSDTRYSPSFKGQVTISADKSISTAYLQWSSL |
| | | KASDTAIYYCVGLEWNYNPLRYWGQGTLVTVSS |
| GL_5-51 | 39 | EVQLVQSGAEVKKPGESLKISCKASGYQLKDNWIGWVRQKPG |
| VH5 | | KGLEWMGIIYPGDSDTRYSPSFQGQVTISADKSISTAYLQWSSL |
| | | KASDTAIYYCVGLEWNYNPLRYWGQGTLVTVSS |
| GL_5-51_ | 40 | EVQLVQSGAEVKKPGESLKISCKASGYQLKDNWIGWVRQKPG |
| VH6 | | KGLEWMGIIYPGDSDTRYSPSFKGQVTISADKSISTAYLQWSSL |
| | | KASDTAIYYCVGLEWNYNPGRYWGQGTLVTVSS |
| GL_5-51 | 41 | EVQLVQSGAEVKKPGESLKISCKASGYQLKDNWAGWVRQKPG |
| VH7 | | KGLEWMGIIYPGDSDTRYSPSFKGQVTISADKSISTAYLQWSSL |
| | | KASDTAIYYCVGLEWNYNPGRYWGQGTLVTVSS |
| GL_5-51_ | 42 | EVQLVQSGAEVKKPGESLKISCKASGYQLKDNWIGWVRQKPG |
| VH8 | | KGLEWMGIIYPGDSDTRYSPSFQGQVTISADKSISTAYLQWSSL |
| | | KASDTAIYYCVGLEWNYNPVRYWGQGTLVTVSS |
| GL_5-51_ | 43 | EVQLVQSGAEVKKPGESLKISCKASGYQLKDNWIGWVRQKPG |
| VH9 | | KGLEWMGIIYPGDSDTRYSPSFQGQVTISADKSISTAYLQWSSL |
| | | KASDTAIYYCVGGEWNYNPLRYWGQGTLVTVSS |
| 1-46_VH1 | 44 | EVQLVQSGAEVKKPGASVKVSCKASGYELNDNWIGWVRQAPG |
| | | QGLEWMGIIYPGDSDTRYAQKFQGRVTMTADKSTSTVYMELSS |
| | | LRSEDTAVYYCVGLEWNYNPLRYWGQGTLVTVSS |
| 1-46_VH2 | 45 | EVQLVQSGAEVKKPGASVKVSCKASGYQLNDNWIGWVRQAPG |
| | | QGLEWMGIIYPGDSDTRYAQKFQGRVTMTADKSTSTVYMELSS |
| | | LRSEDTAVYYCVGLEWNYNPLRYWGQGTLVTVSS |
| 1-46_VH3 | 46 | EVQLVQSGAEVKKPGASVKVSCKASGYELKDNWIGWVRQAPG |
| | | QGLEWMGIIYPGDSDTRYAQKFQGRVTMTADKSTSTVYMELSS |
| | | LRSEDTAVYYCVGLEWNYNPLRYWGQGTLVTVSS |
| 1-46_VH4 | 47 | EVQLVQSGAEVKKPGASVKVSCKASGYQLKDNWAGWVRQAP |
| | | GQGLEWMGIIYPGDSDTRYAQKFQGRVTMTADKSTSTVYMEL |
| | | SSLRSEDTAVYYCVGLEWNYNPLRYWGQGTLVTVSS |
| 1-46_VH5 | 48 | EVQLVQSGAEVKKPGASVKVSCKASGYQLKDNWIGWVRQAPG |
| | | QGLEWMGIIYPGDSDTRYAQKFQGRVTMTADKSTSTVYMELSS |
| | | LRSEDTAVYYCVGLEWNYNPLRYWGQGTLVTVSS |
| 1-46_VH6 | 49 | EVQLVQSGAEVKKPGASVKVSCKASGYQLKDNWIGWVRQAPG |
| | | QGLEWMGIIYPGDSDTRYAQKFQGRVTMTADKSTSTVYMELSS |
| | | LRSEDTAVYYCVGLEWNYNPGRYWGQGTLVTVSS |
| 1-46_VH7 | 50 | EVQLVQSGAEVKKPGASVKVSCKASGYQLKDNWAGWVRQAP |
| | | GQGLEWMGIIYPGDSDTRYAQKFQGRVTMTADKSTSTVYMEL |
| | | SSLRSEDTAVYYCVGLEWNYNPGRYWGQGTLVTVSS |
| 1-46_VH8 | 51 | EVQLVQSGAEVKKPGASVKVSCKASGYQLKDNWIGWVRQAPG |
| | | QGLEWMGIIYPGDSDTRYAQKFQGRVTMTADKSTSTVYMELSS |
| | | LRSEDTAVYYCVGLEWNYNPVRYWGQGTLVTVSS |
| 1-46_VH9 | 52 | EVQLVQSGAEVKKPGASVKVSCKASGYQLKDNWIGWVRQAPG |
| | | QGLEWMGIIYPGDSDTRYAQKFQGRVTMTADKSTSTVYMELSS |
| | | LRSEDTAVYYCVGGEWNYNPLRYWGQGTLVTVSS |
| GL_1-51_VL | 53 | QSVLTQPPSVSAAPGQKVTISCSGSSSDIGSNYVSWYQQLPGT |
| | | APKLLIYDNNKRPSGIPDRFSGSKSGTSATLGITGLQTGDEADY |
| | | YCGTWDSRLGIAVFGGGTQLTVL |
| GL_1-51_VL1 | 54 | QSVLTQPPSVSAAPGQKVTISCSGSSTNIGALWVSWYQQLPGT |
| | | APKLLIYDNNKRPSGIPDRFSGSKSGTSATLGITGLQTGDEADY |
| | | YCATWDSRFGIAVFGGGTQLTVL |
| GL_1-51_VL2 | 55 | QSVLTQPPSVSAAPGQKVTISCSGSSTNIGALWVSWYQQLPGT |
| | | APKLLIYDNNKRPSGIPDRFSGSKSGTSATLGITGLQTGDEADY |
| | | YCATWDSRWGIAVFGGGTQLTVL |
| GL_1-51_VL3 | 56 | QSVLTQPPSVSAAPGQKVTISCSGSSTNIGANYVSWYQQLPGT |
| | | APKLLIYDNNKRPSGIPDRFSGSKSGTSATLGITGLQTGDEADY |
| | | YCATWDSRFGIAVFGGGTQLTVL |
| GL_1-51_VL4 | 57 | QSVLTQPPSVSAAPGQKVTISCSGSSTNIGANYVSWYQQLPGT |
| | | APKLLIYDNNKRPSGIPDRFSGSKSGTSATLGITGLQTGDEADY |
| | | YCATWDSRWGIAVFGGGTQLTVL |
| GL_1-51_VL5 | 58 | QSVLTQPPSVSAAPGQKVTISCSGSSTDIGANYVSWYQQLPGT |
| | | APKLLIYDNNKRPSGIPDRFSGSKSGTSATLGITGLQTGDEADY |
| | | YCATWDSRWGIAVFGGGTQLTVL |
| GL_1-51_VL6 | 59 | QSVLTQPPSVSAAPGQKVTISCSGSSTNIGANYVSWYQQLPGT |
| | | APKLLIYDNQKRPSGIPDRFSGSKSGTSATLGITGLQTGDEADY |
| | | YCATWDSRWGIAVFGGGTQLTVL |
| 1-40_VL1 | 60 | QSVLTQPPSVSGAPGQRVTISCSGSSTNIGALWVSWYQQLPGT |
| | | APKLLIYDNNKRPSGVPDRFSGSKSGTSASLAITGLQAEDEADY |
| | | YCATWDSRFGIAVFGGGTKLTVL |
| 1-40_VL2 | 61 | QSVLTQPPSVSGAPGQRVTISCSGSSTNIGALWVSWYQQLPGT |
| | | APKLLIYDNNKRPSGVPDRFSGSKSGTSASLAITGLQAEDEADY |
| | | YCATWDSRWGIAVFGGGTKLTVL |
| 1-40_VL3 | 62 | QSVLTQPPSVSGAPGQRVTISCSGSSTNIGANYVSWYQQLPGT |
| | | APKLLIYDNNKRPSGVPDRFSGSKSGTSASLAITGLQAEDEADY |
| | | YCATWDSRFGIAVFGGGTKLTVL |
| 1-40_VL4 | 63 | QSVLTQPPSVSGAPGQRVTISCSGSSTNIGANYVSWYQQLPGT |
| | | APKLLIYDNNKRPSGVPDRFSGSKSGTSASLAITGLQAEDEADY |
| | | YCATWDSRWGIAVFGGGTKLTVL |
| 1-40_VL5 | 64 | QSVLTQPPSVSGAPGQRVTISCSGSSTDIGANYVSWYQQLPGT |
| | | APKLLIYDNNKRPSGVPDRFSGSKSGTSASLAITGLQAEDEADY |
| | | YCATWDSRWGIAVFGGGTKLTVL |
| 1-40_VL6 | 65 | QSVLTQPPSVSGAPGQRVTISCSGSSTNIGANYVSWYQQLPGT |
| | | APKLLIYDNQKRPSGVPDRFSGSKSGTSASLAITGLQAEDEADY |
| | | YCATWDSRWGIAVFGGGTKLTVL |

TABLE 1C
CDR SEQUENCES FOR TABLE 1B VH/VL SEQUENCES

| | SEQ ID | |
|---|---|---|
| Clone | NO | Sequence |
| GL_5-51_VH1 | 66 | GYELNDNWIG |
| HCDR1 | | |
| GL_5-51_VH2 | 67 | GYQLNDNWIG |
| HCDR1 | | |
| GL_5-51_VH3 | 68 | GYELKDNWIG |
| HCDR1 | | |
| GL_5-51_VH4 | 69 | GYQLKDNWAG |
| HCDR1 | | |
| GL_5-51_VH5 | 70 | GYQLKDNWIG |
| HCDR1 | | |
| GL_5-51 | 71 | IIYPGDSDTRYSPSFKG |
| HCDR2 | | |
| GL_5-51 | 72 | VGLEWNYNPLRY |
| HCDR3 | | |
| GL_5-51_VH6, | 73 | VGLEWNYNPGRY |
| VH7 HCDR3 | | |
| GL_5-51_VH8 | 74 | VGLEWNYNPVRY |
| HCDR3 | | |
| GL_5-51_VH9 | 75 | VGGEWNYNPLRY |
| HCDR3 | | |
| GL_1-51_VL1, | 76 | SGSSTNIGALWVS |
| VL2 LCDR1 | | |
| GL_1-51_VL3 | 77 | SGSSTNIGANYVS |
| LCDR1 | | |
| GL_1-51_VL5 | 78 | SGSSTDIGANYVS |
| LCDR1 | | |
| GL_1-51_VL6 | 79 | DNQKRPS |
| LCDR2 | | |
| GL_1-51_VL1 | 80 | ATWDSRFGIAV |
| LCDR3 | | |
| GL_1-51_VL2 | 81 | ATWDSRWGIAV |
| LCDR3 | | |
| GL_ 5-51_VH | 82 | VGLDWNYDPLRY |
| MUT1 HCDR3 | | |
| GL_5-51_VH | 83 | VGLEWNYNPLRY |
| MUT2 HCDR3 | | |
| GL_5-51_VH | 84 | GYELTDNWIG |
| MUT3 HCDR1 | | |
| GL_5- | 85 | GYQLTDNWIG |
| 51_VH_MUT4 | | |
| HCDR1 | | |
| GL_5- | 86 | GYSLNDNWIG |
| 51_VH_MUT5 | | |
| HCDR1 | | |
| GL_5- | 87 | GYSLKDNWIG |
| 51_VH_MUT6 | | |
| HCDR1 | | |
| GL_5- | 88 | GYSLTDNWTG |
| 51_VH_MUT8 | | |
| HCDR1 | | |
| GL_5-51_VH | 89 | IIYPGDSDTRYSPSFKGQ |
| _MUT9 | | |
| HCDR2 | | |
| GL_5- | 90 | VGTDWNYNPLRY |
| 51_VH_MUT1 | | |
| 0 HCDR3 | | |
| GL_1- | 91 | GTWDSRFGIAV |
| 51_VL_MUT2 | | |
| LCDR3 | | |
| GL_1- | 92 | SGSSSDIGANYVS |
| 51_VL_MUT3 | | |
| LCDR1 | | |
| GL 1- | 93 | GTWDSRLGIAV |
| 51_VL_MUT3 | | |
| LCDR3 | | |
| GL 1- | 94 | SGSSSDIGSLYVS |
| 51_VL_MUT4 | | |
| LCDR1 | | |
| GL_1- | 95 | GTWDSRLGIAV |
| 51_VL_MUT4, | | |
| MUT5 LCDR3 | | |
| GL_1- | 96 | SGSSTDIGSNYVS |
| 51_VL_MUT5 | | |
| LCDR1 | | |
| GL 1- | 97 | SGSSSDIGSNWVS |
| 51_VL_MUT6 | | |
| LCDR1 | | |
| GL_1- | 98 | SGSSSNIGSNYVS |
| 51_VL_MUT7 | | |
| _LCDR1 | | |
| GL_1- | 99 | ATWDSRLGIAV |
| 51_VL_MUT8 | | |
| LCDR3 | | |

The TSHR antibodies recited in Table 1B and 1C are described in further detail in PCT/US2025/023217, filed Apr. 4, 2025 and incorporated herein by reference.

TABLE 1D
PROTEIN SEQUENCES FOR TSHR ANTIBODY VARIABLE REGION

| | SEQ | |
|---|---|---|
| Antibody | ID NO | Sequence |
| ETY-1 | 100 | GYSLTDNW |
| HCDR1 | | |
| ETY-1 | 101 | IYPGDSDT |
| HCDR2 | | |
| ETY-1 | 102 | VGLDWNYNPLRY |
| HCDR3 | | |
| ETY-1 LCDR1 | 103 | LSDIGSNY |
| ETY-1 LCDR2 | 104 | DNNKRPS |
| ETY-1 LCDR3 | 105 | GTWDSRLGIAV |
| ETY-1 VH | 106 | EVQLVQSGAEVKKPGESLKISCKASGYSLTDNWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSS |
| ETY-1 VL | 107 | QSVLTQPPSVSAAPGQKVTISCSGSLSDIGSNYVSWYQQLP |
| | | GTAPKLLIYDNNKRPSGIPDRFSGSKSGTSATLGITGLQTGD |
| | | EADYYCGTWDSRLGIAVFGGGTQLTVL |
| ETY-2 | 108 | GYSLTDNW |
| HCDR1 | | |
| ETY-2 | 109 | IYPGDSDT |
| HCDR2 | | |
| ETY-2 | 110 | VGLDWNYNPLRY |
| HCDR3 | | |
| ETY-2 LCDR1 | 111 | LRDIGSNY |
| ETY-2 LCDR2 | 112 | DNNKRPS |
| ETY-2 LCDR3 | 113 | GTWDSRLGIAV |
| ETY-2 VH | 114 | EVQLVQSGAEVKKPGESLKISCKASGYSLTDNWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSS |
| ETY-2 VL | 115 | QSVLTQPPSVSAAPGQKVTISCSGSLRDIGSNYVSWYQQLP |
| | | GTAPKLLIYDNNKRPSGIPDRFSGSKSGTSATLGITGLQTGD |
| | | EADYYCGTWDSRLGIAVFGGGTQLTVL |
| ETY-94 | 116 | GYSLTDNW |
| HCDR1 | | |
| ETY-3 | 117 | IYPGDSTT |
| HCDR2 | | |
| ETY-3 | 118 | VGLDWNYNPLRY |
| HCDR3 | | |
| ETY-3 LCDR1 | 119 | YSDIGSNY |
| ETY-3 LCDR2 | 120 | DNNKRPS |
| ETY-3 LCDR3 | 121 | GTWDSRLGIAV |
| ETY-3 VH | 122 | EVQLVQSGAEVKKPGASVKVSCKASGYSLTDNWIGWVRQ |
| | | APGQGLEWMGIIYPGDSTTRYAQKFQGRVTMTADKSTSTV |
| | | YMELSSLRSEDTAVYYCVGLDWNYNPLRYWGQGTLVTVS |
| | | S |
| ETY-3 VL | 123 | QSVLTQPPSVSGAPGQRVTISCSGSYSDIGSNYVSWYQQLP |
| | | GTAPKLLIYDNNKRPSGVPDRFSGSKSGTSASLAITGLQAE |
| | | DEADYYCGTWDSRLGIAVFGGGTKLTVL |
| ETY-4 | 124 | GYSLTDNW |
| HCDR1 | | |
| ETY-4 | 125 | IYPGDSDT |
| HCDR2 | | |
| ETY-4 | 126 | VGLDWNYNPLRY |
| HCDR3 | | |
| ETY-4 LCDR1 | 127 | LRDIGSNY |
| ETY-4 LCDR2 | 128 | DNNKRPS |
| ETY-4 LCDR3 | 129 | GTWDSRLGIAV |
| ETY-4 VH | 130 | EVQLVQSGAEVKKPGASVKVSCKASGYSLTDNWIGWVRQ |
| | | APGQGLEWMGIIYPGDSDTRYAQKFQGRVTMTADKSTSTV |
| | | YMELSSLRSEDTAVYYCVGLDWNYNPLRYWGQGTLVTVS |
| | | S |
| ETY-4 VL | 131 | QSVLTQPPSVSGAPGQRVTISCSGSLRDIGSNYVSWYQQLP |
| | | GTAPKLLIYDNNKRPSGVPDRFSGSKSGTSASLAITGLQAE |
| | | DEADYYCGTWDSRLGIAVFGGGTKLTVL |
| ETY-5 | 132 | GYSLTDNW |
| HCDR1 | | |
| ETY-5 | 133 | IYPGDSDT |
| HCDR2 | | |
| ETY-5 | 134 | VGLDWNYNPLRY |
| HCDR3 | | |
| ETY-5 LCDR1 | 135 | SSNVGSNY |
| ETY-5 LCDR2 | 136 | DNNYVPS |
| ETY-5 LCDR3 | 137 | GTWDSRLDAAV |
| ETY-5 VH | 138 | EVQLVQSGAEVKKPGASVKVSCKASGYSLTDNWIGWVRQ |
| | | APGQGLEWMGIIYPGDSDTRYAQKFQGRVTMTADKSTSTV |
| | | YMELSSLRSEDTAVYYCVGLDWNYNPLRYWGQGTLVTVS |
| | | S |
| ETY-5 VL | 139 | QSVLTQPPSVSGAPGQRVTISCSGSSSNVGSNYVSWYQQLP |
| | | GTAPKLLIYDNNYVPSGVPDRFSGSKSGTSASLAITGLQAE |
| | | DEADYYCGTWDSRLDAAVFGGGTKLTVL |

TABLE 2
HEAVY CHAIN SEQUENCES

| Clone | CDRH1 | CDRH2 | CDRH3 |
|---|---|---|---|
| K1-70 | DNWIG | IIYPGDSDTRYSPSFQG | LDWNYNPLRY |
| | 3 | 4 | 5 |

TABLE 3
LIGHT CHAIN SEQUENCES

| Clone | CDRL1 | CDRL2 | CDRL3 |
|---|---|---|---|
| K1-70 | SGSSSDIGSNYVS | DNNKRPS | GTWDSRLGIAV |
| | 6 | 7 | 8 |

TABLE 4
PROTEIN SEQUENCES FOR IGF-1R
ANTIBODY VARIABLE REGION

| Clone | Seq ID | Chain | Variable Sequence Region |
|---|---|---|---|
| | 9 | heavy | QVELVESGGGWVQPGRSQRLSCA |
| | | | ASGFTFSSYGMHWVRQAPGKGLE |
| | | | WVAIIWFDGSSTYYADSVRGRFT |
| | | | ISRDNSKNTLYLQMNSLRAEDTA |
| | | | VYFCARELGRRYFDLWGRGTLVS |
| | | | VSS |
| TEP | 10 | light | EIVLTQSPATLSLSPGERATLSC |
| | | | RASQSVSSYLAWYQQKPGQAPRL |
| | | | LIYDASKRATGIPARFSGSGSGT |
| | | | DFTLTISSLEPEDFAVYYCQQRS |
| | | | KWPPWTFGQGTKVESK |

TABLE 5
HEAVY CHAIN SEQUENCES

| Clone | CDR11 | CDRH2 | CDRH3 |
|---|---|---|---|
| TEP | SYGMH | IIWFDGSSTYYADSVRG | ELGRRYFDL |
| | 11 | 12 | 13 |

TABLE 6
LIGHT CHAIN SEQUENCES

| Clone | CDRL1 | CDRL2 | CDRL3 |
|---|---|---|---|
| TEP | RASQSVSSYLA | DASKRAT | QQRSKWPPWT |
| | 14 | 15 | 16 |

The following examples are intended to illustrate the invention and are not to be construed as being limitations thereon.

Abbreviations used are those conventional in the art. If not defined, the terms have their generally accepted meanings.

Abbreviations and acronyms used herein include the following:
Expi293 Human Embryonic Kidney (HEK293 High density/serum free) cells
bp base pairs
° C. Centigrade
MEM Minimal Essential Medium
DLS Dynamic light scattering
DNA Deoxyribonucleic acid
ELISA Enzyme linked immuno-adsorbent assay
EC50 Concentration of antibody providing half-maximal response
ECD extracellular domain
g grams
HRP Horseradish peroxidase
IgG Immunoglobulin-G
LIC Ligase independent cloning
min minute
MALS Multi-angle light scattering
nm nanometre
OD optical density
PBS Phosphate Buffered Saline
PCR Polymerase chain reaction
RT Room Temperature
s second
SEC Size exclusion chromatography
TMB 3,3′,5,5′-tetramethylbenzidine
UV Ultra Violet

Nucleotides:

| | |
|---|---|
| A | Adenine |
| C | Cytosine |
| G | Guanine |
| T | Thymine |
| K | G or T (IUPAC convention) |
| M | A or C (IUPAC convention) |
| R | A or G (IUPAC convention) |
| S | C or G (IUPAC convention) |
| V | A or C or G (IUPAC convention) |
| W | A or T (IUPAC convention) |
| Y | C or T (IUPAC convention) |

Amino acids:

| | | | |
|---|---|---|---|
| Ala (A) | Alanine | Met (M) | Methionine |
| Cys (C) | Cysteine | Asn (N) | Asparagine |
| Asp (D) | Aspartic acid | Pro (P) | Proline |
| Glu (E) | Glutamic acid | Gln (Q) | Glutamine |
| Phe (F) | Phenylalanine | Arg (R) | Arginine |
| Gly (G) | Glycine | Ser (S) | Serine |
| His (H) | Histidine | Thr (T) | Threonine |
| Ile (I) | Isoleucine | Val (V) | Valine |
| Lys (K) | Lysine | Trp (W) | Tryptophan |
| Leu (L) | Leucine | Tyr (Y) | Tyrosine |

All publications, patents and patent applications herein are incorporated by reference to the same extent as if each individual publication or patent application was specifically and individually indicated to be incorporated by reference. The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art. Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### EXAMPLES

### Example 1

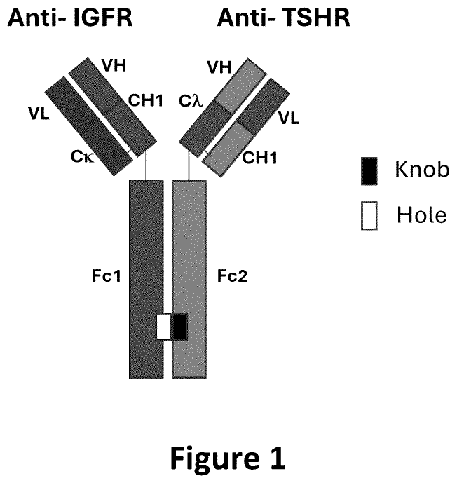

A bispecific antibody of the invention, Example 1 (K1-70+Teprotumumab), was prepared using CrossMab^{CH-CL} technology described hereinabove wherein the CH1 and Cλ constant regions of the anti-TSHR arm were crossed as shown in FIG. 1. The amino acid sequences are described below with reference to FIG. 1.

### Amino Acid Sequences

### Anti-IGF-1R Heavy Chain Sequence: VH-CH1-Fc1 SEQ ID NO 31

VH as set forth in Table 4
CH1 Sequence SEQ ID NO: 18
Fc1 Sequence SEQ ID NO: 28

### Anti-IGF-1R Light Chain Sequence: VL-Cκ SEQ ID NO 32

VL as set forth in Table 4
Cκ Sequence SEQ ID NO: 20

### Anti-TSHR Heavy Chain Sequence: VH-Cλ-Fc2 SEQ ID NO:29

VH as set forth in Table 1
Cλ Sequence SEQ ID NO: 23
Fc2 Sequence SEQ ID NO: 27

### Anti-TSHR Light Chain Sequence: VL-CH1 SEQ ID NO:30

VL as set forth in Table 1
CH1 Sequence SEQ ID NO: 22

### Example 2: IGF-1R ELISA Binding Assay

Antigen was coated onto an ELISA plate at 1 μg/mL (100 μL/well) at 4° C. overnight. Next, plates were blocked with 2% BSA (200 μL/well) at room temperature for 1 hour. Test antibodies were serially diluted with a 4-fold dilution starting at a concentration of 400 nM to form an 11-point curve. Subsequently 100 μL of each test antibody dilution series was added to the appropriate well of the ELISA plate which was then incubated at room temperature for 2 hours. Next, goat anti-human IgG Fc-HRP was added to every well of the plate (100 μL/well) and incubated at room temperature for 1 hour. Plates were then developed with TMB substrate and subsequently stopped with Sulphuric Acid. Finally, Absorbance was read at 450 and 540 nm by Mse.

Bispecific antibody Example 1 (K1-70+Teprotumumab) and Teprotumumab mAb both show dose-dependent binding to IGF1R. K1-70 mAb did not bind significantly to IGF1R.

### Example 3: TSHR ELISA Binding Assay

Rabbit Anti His Antibody was coated onto an ELISA plate at 1 μg/mL (100 μL/well) at 4° C. overnight. Next, plates were blocked with 2% BSA (200 μL/well) at room temperature for 1 hour. 2 μg/mL Antigen was prepared in Blocking Buffer and added to every well of the plate (100 μL/well). Test antibodies were serially diluted with a 4-fold dilution starting at a concentration of 400 nM to form an 11-point curve. Subsequently, 100 μL of each test antibody dilution series was added to the appropriate well of the ELISA plate which was then incubated at room temperature for 2 hours. Next, goat anti-human IgG Fc-HRP was added to every well of the plate (100 μL/well) and incubated at room temperature for 1 hour. Plates were then developed with TMB substrate and subsequently stopped with Sulphuric Acid. Finally, Absorbance was read at 450 and 540 nm by Mse. Bispecific antibody Example 1 (K1-70+Teprotumumab) and K1-70 mAb both showed dose-dependent binding to TSHR. Teprotumumab mAb did not bind significantly to TSHR,

### Example 4: FACS (Fluorescence Activated Cell Sorting)

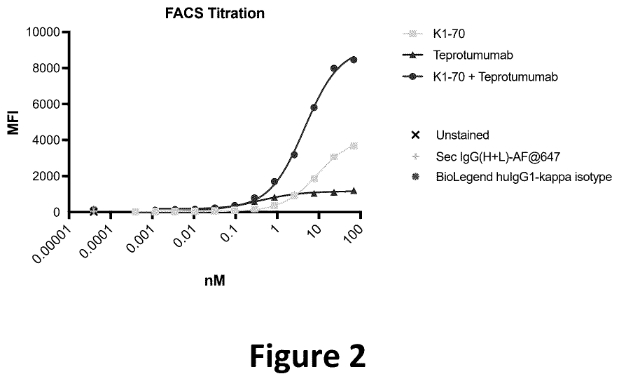

The HiTSeeker HEK293-Human-TSHR cell line was grown in DMEM medium supplemented with 10% FCS, 1% Pen-Strep and 0.4 mg/mL Hygromycin B Gold (Invitrogen; #ant-hg-1) at 37° C., 5% CO₂. Cells were detached from the flask using Accutase (eBioscience) for 5-10 min at 37° C., 5% CO₂ then 0.5×10⁵ cells/sample were incubated in PBS supplemented with 2% FCS (FACS buffer) for 10 min at 4° C. Samples were washed in FACS buffer then centrifuged at 1500 rpm for 3 min. Next, a serial dilution of 0-10 μg/mL of primary antibody was prepared and added to the samples then incubated for 15 min at 4° C. Cells were washed twice in FACS buffer. Next, secondary goat anti-human IgG (H+L)—Alexa Fluor 647 antibody (ThermoFisher #A21445, 1/1000 dilution) was added to samples and incubated for 15 min at 4° C. Samples were washed twice in FACS buffer. Finally, samples were acquired on a Navios flow cytometer (Beckman Coulter) and data were analysed using FlowJo software (BD Biosciences). As shown in FIG. 2 and Table 7 below, using the above FACS protocol the EC50 for the bispecific antibody Example 1 (K1-70+Teprotumumab) was determined to be 4.55 nM, in comparison to K1-70 mAb (9.01 nM) teprotumumab mAb (0.32 nM).

TABLE 7
FACS data

| Antibody | EC50 (nM) |
|---|---|
| K1-70 | 9.01 |
| Teprotumumab | 0.32 |
| K1-70 + Teprotumumab | 4.55 |

Bispecific antibody Example 1 (K1-70+Teprotumumab), K1-70 mAb and Teprotumumab mAb all show dose dependent binding to the co-expressing cells as expected. In this example, the bispecific antibody Example 1 (K1-70+Teprotumumab) has a lower EC50 than either K1-70 mAb or Teprotumumab mAb. Bispecific antibody Example 1 (K1-70+Teprotumumab) also has the highest maximum MFI, suggesting that the bispecific antibody Example 1 (K1-70+Teprotumumab targeting both TSHR and IGF-1R exhibits increased binding and at higher density to the target co-expressing cell

### Example 5: LigandTracer Live Cell Kinetics

Cell dishes were prepared with HEK293-TSHR cells or parental HEK293 cells in CO₂ independent media (Gibco 18045) supplemented with 2 mM L-Glutamine (Biowest X0550). Prepared cell dishes were placed in the LigandTracer instrument (LigandTracer Green (Ridgeview Instruments AB) equipped with a Blue (ex. 488 nm)-Green (em. 535 nm) detector) on an inclined and rotating cell dish holder and a baseline was recorded. During the association phase, two increasing concentrations of ligand i.e., Atto488-labeled antibodies of interest, were added. The association phase was followed by a dissociation phase for which the incubation solution was replaced with fresh CO₂-independent medium without ligand. During each rotation of the cell dish, the fluorescence signal from both target cell spots and both reference spots, not containing any cells, was detected resulting in two reference subtracted binding curves. The detection time was 15 s per spot, the detection delay time was set to 3 s per spot and the rotation speed was set to “normal” for all experiments. Data was collected by the instrument control software LigandTracer Control version 2.6 (Ridgeview Instruments AB). Subsequently real-time interaction data was analysed and kinetic parameters were extracted with TraceDrawer Version 1.9.2 (Ridgeview Instruments AB). Real time binding kinetics were modelling by using a 1:1 fit model, or the 1:1 fit Bulk Index.

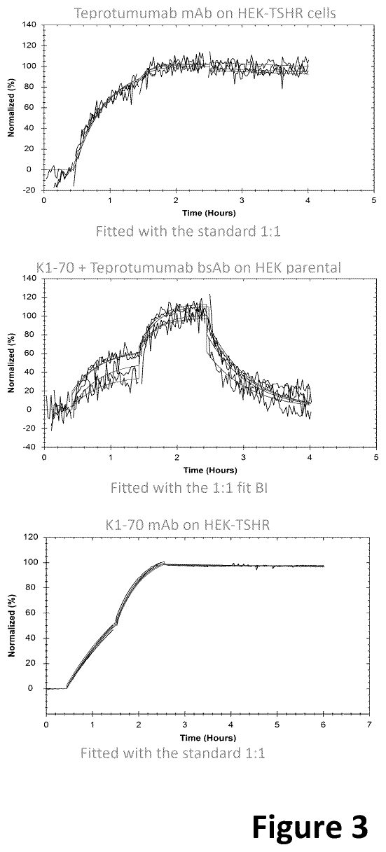

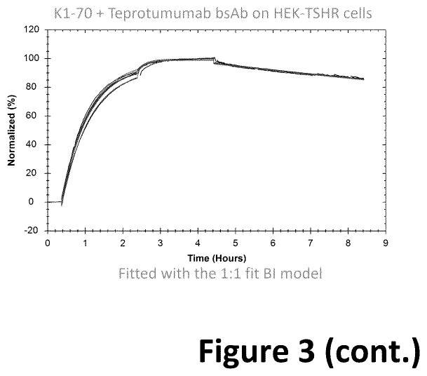

As shown in FIG. 3 and Table 8 below, using the above protocol the Kd (M) values for the bispecific antibody Example 1 (K1-70+Teprotumumab), K1-70 mAb, and teprotumumab mAb were determined.

TABLE 8
LigandTracer Live Cell Kinetics Data

| Antibody | Cell | Targeted | Ka | Kd | KD |
|---|---|---|---|---|---|
| Construct | Line | Receptor | [1/(M*s)] | (1/s) | (M) |
| Teprotumumab | HEK- | IGF-1R | 3.3E+05 | 1.2E−03 | 3.7E−09 |
| single arm | TSHR | | | | |
| Teprotumumab | HEK- | IGF-1R | 5.6E+05 | 6.9E−06 | 1.2E−11 |
| mAb | TSHR | | | | |
| K1-70 single | HEK- | TSHR | 1.7E+05 | 7.7E−06 | 4.5E−10 |
| arm | TSHR | | | | |
| K1-70 mAb | HEK- | TSHR | 3.3E+05 | 1.4E−06 | 4.3E−11 |
| | TSHR | | | | |
| K1-70 + | Parental | IGF-1R | 2.0E+05 | 4.5E−04 | 2.2E−09 |
| Teprotumumab | HEK | | | | |
| BsAb | | | | | |
| K1-70 + | HEK- | IGF-1R | 3.7E+04 | 8.6E−06 | 2.3E−10 |
| Teprotumumab | TSHR | and | | | |
| BsAb | | TSHR | | | |

The bispecific antibody Example 1 (K1-70+Teprotumumab) binds to IGF-1R only expressing cells with a significantly lower affinity (2.2 nM) compared to when both targets are expressed (0.23 nM). This shows that both arms of the bispecific molecule can bind to both cell surface targets and the antibody benefits from avidity on the target IGF1R+TSHR+GOF cells.

The bispecific antibody Example 1 (K1-70+Teprotumumab) binds to IGF-1R only expressing cells significantly more weakly (2.2 nM) than Teprotumumab (12 μM) This greater than 180 fold difference suggests a significantly lower binding to peripheral IGF-1R only expressing cells

In this context, the IGF1R only expressing cells may represent peripheral IGF-1R expressing (TSHR-′ve) cells, and the co-expressing cells may represent the target GOF and thyroid cells. Given the high affinity of the bispecific antibody Example 1 (K1-70+Teprotumumab) for the TSHR and IGF1R co-expressing cells (0.23 nM), a skilled person would expect bispecific antibody Example 1 (K1-70+Teprotumumab) to have significantly less binding to IGF1R+ TSHR− cells than Teprotumumab whilst retaining very effective (sub-nanomolar) binding to TSHR+ IGF1R+ target GOF cells. The data therefore suggest a significantly advantageous drug distribution, with lower off-tissue related negative side-effects.

### Example 6: HA Assay

Graves' Orbital Fibroblasts (GOFs) were isolated from retro-orbital connective tissue which was obtained from patients with severe TED undergoing orbital decompression surgery. Cells were cultured in DMEM with FBS (10% vol/vol), penicillin (100 U/mL), streptomycin (100 μg/mL), L-glutamine (2 mM), Ham's F-12 nutrient mixture (25% vol/vol), hydrocortisone (25 ng/mL), epithelial growth factor (0.125 ng/ml), insulin (5 μg/mL), cholera toxin (11.7 nM), gentamicin (10 μg/mL), amphotericin B (250 ng/mL), and Y-27632 (5 UM) and maintained at 37° C. in a 7% CO₂ humidified incubator.

Serum samples were obtained from patients with phenotypically overt and clinically severe TED. IgG antibodies were isolated from whole serum (TED-IgGs) by thiophilic affinity chromatography and subsequently pooled.

TEDOFs were plated at confluence (4×10⁵ cells/cm²) in 48-well plates and stimulated with M22 or TED-IgGs with increasing doses of K1-70 mAb, Teprotumumab mAb or K1-70+Teprotumumab bsAb in DMEM containing 10% FBS and 10 mM HEPES. Cells were incubated at 37° C., 7% CO₂, for 5 days. Conditioned media was collected and stored at −20° C. HA concentrations were measured by a modified Corgenix HA ELISA in which the kit supplied HA Standard was replaced with freshly prepared 1 million molecular weight HA as described in (Kreiger and Gershengorn, 2014).

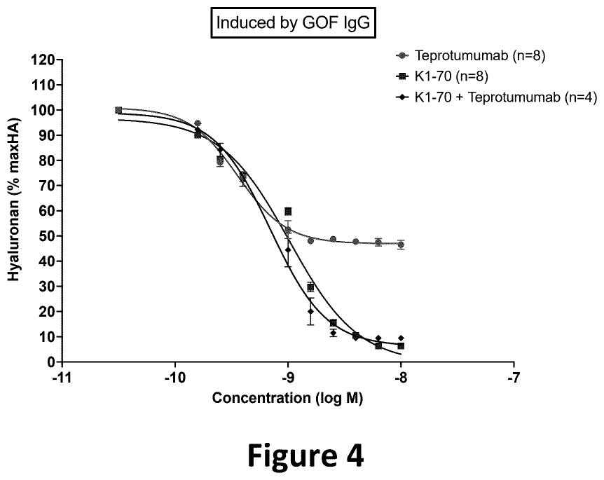

As shown in FIG. 4 and Table 9 below, using the above protocol the IC50 values for the bispecific antibody Example 1 (K1-70+Teprotumumab), K1-70 mAb and teprotumumab mAb, were determined.

TABLE 9
HA Assay Data

| Antibody | IC50 (nM) |
|---|---|
| K1-70 | 1.027 |
| Teprotumumab | 0.358 |
| K1-70 + Teprotumumab | 0.792 |

Thyroid eye disease (TED) is driven by autoantibodies to TSHR. This activates the TSHR pathway, which leads to increased production of HA and remodelling of orbital tissue. Crosstalk occurs incidentally through activation of IGF1R pathway, enhancing inflammatory signalling and HA production (Krieger C C, Neumann S, Gershengorn M C. TSH/IGF1 receptor crosstalk: Mechanism and clinical implications. Pharmacol Ther. 2020 May; 209:107502, incorporated herein by reference). Whilst not bound by theory, inhibition of IGF-1R only reduces the crosstalk signalling and does not block the TSHR pathway signalling. Hence blocking IGF-1R crosstalk reduces HA production by only ~50% This leaves significant room for therapeutic improvement.

In this assay it is shown that blocking TSHR and IGF1R with bispecific antibody Example 1 (K1-70+Teprotumumab) reduces HA production by 100%, which is equivalent to K1-70 mAb and 100% more effective than blocking IGF1R alone with Teprotumumab. This level of activity in the HA assay may be expected to result greater improvements in CAS scores and proptosis and quality of life for patients.

At the same time the K1-70+Teprotumumab bispecific demonstrates ~180× lower binding to IGF-1R+ TSHR− ′ve cells compared to Teprotumumab, while demonstrating sub-nanomolar binding to TSHR+ IGF1R+ co-expressing target cells.

This indicates that the bispecific will have total normalization of TSHR stimulating antibody HA production while having significantly lower binding to IGF1R+ only cells, which would be expected to translate to fewer on-target adverse events compared to IGF1R mAbs with significantly higher IGF-1R avidity.

### Example 7: cAMP Assay

The HiTSeeker TSHR/HEK293 cell line was grown in DMEM medium supplemented with 10% FCS, 1% Pen-Strep and 0.25 mg/mL G418 (Geneticin, Gibco #10131-035) at 37° C., 5% CO₂. 6000 cells per well were seeded into 384-well LV plate (Greiner #3284). The cells were incubated with increasing concentrations of test antibodies, from 0-2.7 μM in HBSS for 15 min at 37° C. and then stimulated with 0.3 nM of M22 (EC₅₀) for 45 min at 37° C. The cells and cAMP standard dilutions were further processed using HTRF CAMP kit according to the manufacturer's instructions.

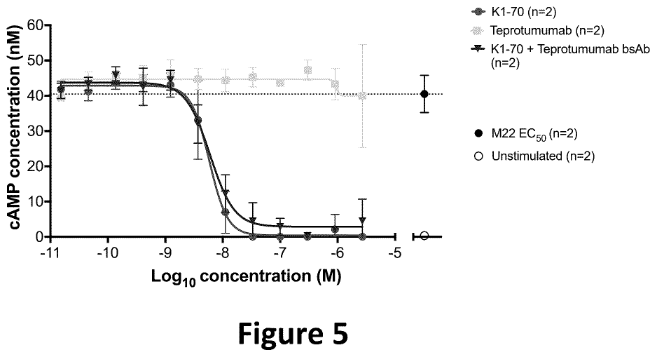

As shown in FIG. 5 and Table 10 below, using the above protocol the IC50 vs M22 values for the bispecific antibody Example 1 (K1-70+Teprotumumab), K1-70 mAb and teprotumumab mAb, were determined.

TABLE 10
cAMP Assay Data

| Antibody | IC50 vs M22 (nM) |
|---|---|
| K1-70 | 5.82 |
| Teprotumumab | N/A |
| K1-70 + Teprotumumab | 5.99 |

In a IGF1R+ TSHR+ cell line, bispecific antibody Example 1 (K1-70+Teprotumumab) inhibits the M22 stimulated TSHR cAMP response equivalently to the K1-70 mAb. Teprotumumab mAb has no inhibitory effect on M22 stimulated TSHR cAMP response.

### Example 8: Additional TSHR Antibodies

Additional TSHR antibodies were generated that may be paired with an IGF-1R antibody to form the multispecific antibodies described herein.

Select TSHR antibody variants were tested for their ability to bind TSHR in an ELISA. Briefly, soluble TSHR protein was coated onto a plate at 2 μg/ml in phosphate buffered saline (PBS). The plate was washed with PBS and blocked with 4% skim milk in PBS.

Three concentrations of test antibody (300, 100 and 33 nM) were plated including the parental antibody and human IgG1 isotype control antibody. Following wash steps with 1×PBS-Tween 0.05%, bound human IgG was detected with peroxidase conjugated goat anti-human IgG (Fc specific)-HRP at 1:5000 dilution and the ELISA was developed with TMB reagent followed by H2SO4. The plate was read by O.D. at 450 nm measured using a microplate spectrophotometer

ELISA data was compared against the parental antibody sequence from which the TSHR antibody variants are derived. ELISA values are reported below in Table 11.

TABLE 11
ELISA Values

| | | | — | — | — | ELISA (OD 450) | | |
|---|---|---|---|---|---|---|---|---|
| — | — | — | ka | kd | KD | 300 | 100 | 33.3 |
| — | VH | VL | (1/s*M) | (1/s) | (nM) | nM | nM | nM |
| parental | parental VH | parental VL | 1.42E+05 | 3.30E−04 | 2.32 | 0.461 | 0.43 | 0.406 |
| TSHB- | GL_5-51 VH | parental VL | 1.61E+05 | 2.73E−04 | 1.7 | 0.641 | 0.506 | 0.442 |
| 1 | | | | | | | | |
| TSHB- | GL_5- | parental VL | 1.98E+05 | 2.73E−03 | 13.8 | 0.081 | 0.072 | 0.069 |
| 2 | 51_VH_MUT1 | | | | | | | |
| TSHB- | GL_5- | parental VL | 1.77E+05 | 5.33E−04 | 3.01 | 0.467 | 0.407 | 0.351 |
| 3 | 51_VH_MUT2 | | | | | | | |
| TSHB- | GL_5- | parental VL | 1.50E+05 | 3.62E−04 | 2.41 | 0.542 | 0.453 | 0.374 |
| 4 | 51_VH_MUT3 | | | | | | | |
| TSHB- | GL_5- | parental VL | 1.56E+05 | 4.40E−04 | 2.82 | 0.578 | 0.498 | 0.414 |
| 5 | 51_VH_MUT4 | | | | | | | |
| TSHB- | GL_5- | parental VL | 1.42E+05 | 5.32E−04 | 3.73 | 0.548 | 0.493 | 0.45 |
| 6 | 51_VH_MUT5 | | | | | | | |
| TSHB- | GL_5- | parental VL | 1.16E+05 | 4.77E−04 | 4.11 | 0.555 | 0.491 | 0.443 |
| 7 | 51_VH_MUT6 | | | | | | | |
| TSHB- | GL_5- | parental VL | 1.53E+05 | 3.44E−04 | 2.25 | 0.586 | 0.551 | 0.471 |
| 8 | 51_VH_MUT7 | | | | | | | |
| TSHB- | GL_5- | parental VL | 1.04E+05 | 6.76E−04 | 6.53 | 0.478 | 0.452 | 0.355 |
| 9 | 51_VH_MUT8 | | | | | | | |
| TSHB- | GL_5- | parental VL | 1.15E+05 | 3.38E−04 | 2.94 | 0.557 | 0.496 | 0.44 |
| 10 | 51_VH_MUT9 | | | | | | | |
| TSHB- | GL_5- | parental VL | 1.21E+04 | 6.43E−04 | 53.3 | 0.289 | 0.22 | 0.159 |
| 11 | 51_VH_MUT10 | | | | | | | |
| TSHB- | 1-46_VH | parental VL | 1.40E+05 | 5.08E−04 | 3.62 | 0.52 | 0.475 | 0.402 |
| 12 | | | | | | | | |
| TSHB- | parental VH | GL_1- | 1.41E+05 | 2.59E−04 | 1.84 | 0.561 | 0.495 | 0.459 |
| 13 | | 51_VL | | | | | | |
| TSHB- | parental VH | GL_1- | 1.40E+05 | 3.52E−04 | 2.52 | 0.572 | 0.522 | 0.477 |
| 14 | | 51_VL_MUT1 | | | | | | |
| TSHB- | parental VH | GL_1- | 1.21E+05 | 3.72E−04 | 3.08 | 0.569 | 0.517 | 0.443 |
| 15 | | 51_VL_MUT2 | | | | | | |
| TSHB- | parental VH | GL_1- | 1.42E+05 | 3.85E−04 | 2.7 | 0.566 | 0.487 | 0.443 |
| 16 | | 51_VL_MUT3 | | | | | | |
| TSHB- | parental VH | GL_1- | 4.94E+04 | 4.07E−04 | 8.25 | 0.475 | 0.402 | 0.357 |
| 17 | | 51_VL_MUT4 | | | | | | |
| TSHB- | parental VH | GL_1- | 1.25E+05 | 3.68E−04 | 2.94 | 0.55 | 0.495 | 0.42 |
| 18 | | 51_VL_MUT5 | | | | | | |
| TSHB- | parental VH | GL_1- | 1.18E+05 | 3.69E−04 | 3.12 | 0.547 | 0.47 | 0.405 |
| 19 | | 51_VL_MUT6 | | | | | | |
| TSHB- | parental VH | GL_1- | 1.09E+05 | 3.01E−04 | 2.77 | 0.54 | 0.465 | 0.411 |
| 20 | | 51_VL_MUT7 | | | | | | |
| TSHB- | parental VH | GL_1-51 | 1.15E+05 | 3.57E−04 | 3.1 | 0.552 | 0.473 | 0.395 |
| 21 | | VL_MUT8 | | | | | | |
| TSHB- | parental VH | 1-40_VL | 1.29E+05 | 3.23E−04 | 2.5 | 0.53 | 0.493 | 0.447 |
| 22 | | | | | | | | |
| TSHB- | GL_5-51 VH | GL_1- | 1.41E+05 | 3.44E−04 | 2.45 | 0.538 | 0.466 | 0.512 |
| 23 | | 51_VL | | | | | | |
| TSHB- | GL_5-51 VH | 1-40_VL | 1.29E+05 | 3.48E−04 | 2.7 | 0.54 | 0.49 | 0.425 |
| 24 | | | | | | | | |
| TSHB- | 1-46_VH | 1-40_VL | 1.38E+05 | 6.16E−04 | 4.48 | 0.367 | 0.409 | 0.38 |
| 25 | | | | | | | | |
| hIgG1 | — | — | — | — | — | 0.055 | 0.057 | 0.063 |
| isotype | | | | | | | | |
| control | | | | | | | | |
| blank | — | — | — | — | — | 0.055 | 0.052 | 0.051 |

Affinity of the TSHR antibodies to their target antigen was also investigated using surface plasmon resonance (SPR), Table 1. Measurements were taken on a Biacore T200 at 25 C using a CM5 chip coated with anti-human Fc. Test antibodies were captured at 0.1 ug/mL in HBS-EP+ (10 mM HEPES pH7.4, 150 mM NaCl, 3 mM EDTA, 0.05% Tween 20) assay buffer. Soluble TSHR (22-260) protein was flowed over the chip at 0 nM, 0.41 nM, 1.24 nM, 3.07 nM, 11.11 nM, 33.33 nM, 100 nM, 300 nM, 900 nM at a flow rate of 30 μL/min with an association time of 90 s and dissociation time of 200 s. A 1:1 binding kinetics fit was used to calculate kinetic parameters.

Affinity for TSHR by the antibodies can be screened using a kinetic exclusion assay (KinExA). K_{D} determinations are most accurate when the concentration of the receptor (e.g., TSHR) in the sample is near or below the K_{D}. Binding curves can also be generated for the antibodies.

### Example 9. Additional TSHR Antibodies

Additional TSHR antibodies were generated that may be paired with an IGF-1R antibody to form the multispecific antibodies described herein.

Affinity of the 5 TSHR antibodies to their target antigen was investigated using surface plasmon resonance (SPR), Table 12. Measurements were taken on a Bruker MASS-2 on a High-Capacity Amine (HCA) Sensor (Bruker #1862614) immobilized with >10,000 RU anti-HuFc IgG1 (MP Bio #0855071) on spots AB. 40 nM of purified test antibody was injected for 45 sec at 25 μL/min, aiming for a capture level of 400 RU. Biotinylated TSHR22-260 Avi-His tag was injected over spots AB for 4 min at 50 μL/min, followed by 30 min dissociation phase. Analyte concentrations of 6.25 nM, 12.5 nM, 25 nM, 50 nM, and 100 nM were injected in MCK-assay format, from lowest to highest. Running buffer was PBS, pH 7.4 with 0.05% v/v Tween 20. Chip was regenerated with 10 mM Glycine-HCl, pH 1.7 injected for 15 seconds at 25 μL/min. Buffer blank injections and reference spot (A) analyte injections are each subtracted to produce double-subtracted sensorgrams. Data was analyzed using Bruker Analyzer 4 Software.

Affinity for TSHR by the antibodies can be screened using a kinetic exclusion assay (KinExA). K_{D} determinations are most accurate when the concentration of the receptor (e.g., TSHR) in the sample is near or below the K_{D}. Binding curves can also be generated for the antibodies.

TABLE 12
Binding Characteristics

| Antibody | kon (M−1 s−1) | koff (s−1) | KD (M) |
|---|---|---|---|
| ETY-1 | 1.21E+05 | 3.56E−05 | 2.95E−10 |
| ETY-2 | 1.15E+05 | 3.05E−05 | 2.66E−10 |
| ETY-3 | 7.96E+04 | 6.27E−05 | 7.87E−10 |
| ETY-4 | 9.97E+04 | 6.20E−05 | 6.21E−10 |
| ETY-5 | 7.21E+04 | 4.91E−05 | 6.81E−10 |
| hIgG1 isotype | — | — | — |
| control | | | |
| blank | — | — | — |

SEQUENCE LISTINGS
SEQ ID NO: 1
EVQLVQSGAEVKKPGQSLKISCKASGYSLTDNWIGWVRQKPGKGLEWMGIIYPGDSDT
RYSPSFQGQVTISADKSINTAYLQWSSLKASDTAIYYCVGLDWNYNPLRYWGPGTLVTV
SS
SEQ ID NO: 2
QSVLTQPPSVSAAPGQKVTISCSGSSSDIGSNYVSWYQQFPGTAPKLLIYDNNKRPSAIP
DRFSGSKSGTSATLGITGLQTGDEADYYCGTWDSRLGIAVFGGGTQLTVL
SEQ ID NO: 3
DNWIG
SEQ ID NO: 4
IIYPGDSDTRYSPSFQG
SEQ ID NO: 5
LDWNYNPLRY
SEQ ID NO: 6
SGSSSDIGSNYVS
SEQ ID NO: 7
DNNKRPS
SEQ ID NO: 8
GTWDSRLGIAV
SEQ ID NO: 9
QVELVESGGGVVQPGRSQRLSCAASGFTFSSYGMHWVRQAPGKGLEWVAIIWFDGSS
TYYADSVRGRFTISRDNSKNTLYLQMNSLRAEDTAVYFCARELGRRYFDLWGRGTLVSV
SS
SEQ ID NO: 10
EIVLTQSPATLSLSPGERATLSCRASQSVSSYLAWYQQKPGQAPRLLIYDASKRATGIPA
RFSGSGSGTDFTLTISSLEPEDFAVYYCQQRSKWPPWTFGQGTKVESK
SEQ ID NO: 11
SYGMH
SEQ ID NO: 12
IIWFDGSSTYYADSVRG
SEQ ID NO: 13
ELGRRYFDL
SEQ ID NO: 14
RASQSVSSYLA
SEQ ID NO: 15
DASKRAT
SEQ ID NO: 16
QQRSKWPPWT
SEQ ID NO: 17
ASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKV
SEQ ID NO: 18
ASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSC
SEQ ID NO: 19
GQPKAAPSVTLFPPSSEELQANKATLVCLISDFYPGAVTVAWKADSSPVKAGVETTTPSK
QSNNKYAASSYLSLTPEQWKSHRSYSCQVTHEGSTVEKTVAPTECS
SEQ ID NO: 20
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 21
SSASTKGPSVFPLAPSSKSTSGGTAALGCLVKDYFPEPVTVSWNSGALTSGVHTFPAVL
QSSGLYSLSSWVTVPSSSLGTQTYICNVNHKPSNTKVDKKV
SEQ ID NO: 22
SSASTKGPSVFPLAPSSKSTSGGTAALGCLVKDYFPEPVTVSWNSGALTSGVHTFPAVL
QSSGLYSLSSWTVPSSSLGTQTYICNVNHKPSNTKVDKKVEPKSC
SEQ ID NO: 23
ASGQPKAAPSVTLFPPSSEELQANKATLVCLISDFYPGAVTVAWKADSSPVKAGVETTTP
SKQSNNKYAASSYLSLTPEQWKSHRSYSCQVTHEGSTVEKTVAPTECS
SEQ ID NO: 24
ASVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
SEQ ID NO: 25
APELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
SEQ ID NO: 26
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
SEQ ID NO: 27
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPENNYKTTPPV
LDSDGSFFLYSKLTVDKSRWQQGNVFSCSVMHEALHNHYTQKSLSLSPG
SEQ ID NO: 28
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTISKA
KGQPREPQVCTLPPSRDELTKNQVSLSCAVKGFYPSDIAVEWESNGQPENNYKTTPPVL
DSDGSFFLVSKLTVDKSRWQQGNVFSCSVMHEALHNHYTQKSLSLSPGK
SEQ ID NO: 29
EVQLVQSGAEVKKPGQSLKISCKASGYSLTDNWIGWVRQKPGKGLEWMGIIYPGDSDT
RYSPSFQGQVTISADKSINTAYLQWSSLKASDTAIYYCVGLDWNYNPLRYWGPGTLVTV
SSASGQPKAAPSVTLFPPSSEELQANKATLVCLISDFYPGAVTVAWKADSSPVKAGVETT
TPSKQSNNKYAASSYLSLTPEQWKSHRSYSCQVTHEGSTVEKTVAPTECSDKTHTCPP
CPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREP
QVYTLPPCRDELTKNQVSLWCLVKGFYPSDIAVEWESNGQPENNYKTTPPVLDSDGSFF
LYSKLTVDKSRWQQGNVFSCSVMHEALHNHYTQKSLSLSPG
SEQ ID NO: 30
QSVLTQPPSVSAAPGQKVTISCSGSSSDIGSNYVSWYQQFPGTAPKLLIYDNNKRPSAIP
DRFSGSKSGTSATLGITGLQTGDEADYYCGTWDSRLGIAVFGGGTQLTVLSSASTKGPS
VFPLAPSSKSTSGGTAALGCLVKDYFPEPVTVSWNSGALTSGVHTFPAVLQSSGLYSLS
SWTVPSSSLGTQTYICNVNHKPSNTKVDKKVEPKSC
SEQ ID NO: 31
QVELVESGGGVVQPGRSQRLSCAASGFTFSSYGMHWVRQAPGKGLEWVAIIWFDGSS
TYYADSVRGRFTISRDNSKNTLYLQMNSLRAEDTAVYFCARELGRRYFDLWGRGTLVSV
SSASTKGPSVFPLAPSSKSTSGGTAALGCLVKDYFPEPVTVSWNSGALTSGVHTFPAVL
QSSGLYSLSSWVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVCTLPP
SRDELTKNQVSLSCAVKGFYPSDIAVEWESNGQPENNYKTTPPVLDSDGSFFLVSKLTV
DKSRWQQGNVFSCSVMHEALHNHYTQKSLSLSPGK
SEQ ID NO: 32
EIVLTQSPATLSLSPGERATLSCRASQSVSSYLAWYQQKPGQAPRLLIYDASKRATGIPA
RFSGSGSGTDFTLTISSLEPEDFAVYYCQQRSKWPPWTFGQGTKVESKRTVAAPSVFIF
PPSDEQLKSGTASWVCLLNNFYPREAKVQWKVDNALQSGNSQESVTEQDSKDSTYSLS
STLTLSKADYEKHKVYACEVTHQGLSSPVTKSFNRGEC

What is claimed is:

1. A multispecific antibody molecule comprising:
(i) at least one anti-thyroid stimulating hormone receptor (TSHR) antibody or antigen-binding domain, which binds to TSHR, and
(ii) at least one anti-insulin like growth factor 1 receptor (IGF-1R) antibody or antigen-binding domain, which binds to IGF-1R,
wherein the at least one anti-TSHR antibody or antigen-binding domain has a higher affinity to TSHR than the at least one anti-IGF-1R antibody or antigen-binding domain having an affinity to IGF-1R.

2. The multispecific antibody molecule of claim 1, wherein the multispecific antibody molecule is a bispecific antibody.

3. The multispecific antibody molecule of claim 1, wherein the at least one anti-TSHR antibody or antigen-binding domain has an at least 5 fold, at least 10 fold, or at least 100 fold higher affinity to TSHR than the at least one anti-IGF-1R antibody or antigen-binding domain having an affinity to IGF-1R as determined by the dissociation equilibrium constant K (Kd).

4. The multispecific antibody molecule of claim 3, wherein the multispecific antibody molecule has a TSHR affinity with dissociation equilibrium constant K (Kd) in the pM range and an IGF-1R affinity in the nM range.

5. The multispecific antibody molecule of claim 1, wherein the at least one anti-TSHR antibody or antigen-binding domain comprises:
(i) a variable heavy chain region (VH) comprising an HCDR1 amino acid sequence of SEQ ID NO: 3, an HCDR2 amino acid sequence of SEQ ID NO: 4, and an HCDR3 amino acid sequence of SEQ ID NO: 5, and a variable light chain region (VL) comprising an LCDR1 amino acid sequence of SEQ ID NO: 6, an LCDR2 amino acid sequence of SEQ ID NO: 7, and an LCDR3 amino acid sequence of SEQ ID NO: 8;
(ii) a VH comprising an HCDR1 amino acid sequence of SEQ ID NO: 100, an HCDR2 amino acid sequence of SEQ ID NO: 101, and an HCDR3 amino acid sequence of SEQ ID NO: 102, and a VL comprising an LCDR1 amino acid sequence of SEQ ID NO: 103, an LCDR2 amino acid sequence of SEQ ID NO: 104, and an LCDR3 amino acid sequence of SEQ ID NO: 105;
(iii) a VH comprising an HCDR1 amino acid sequence of SEQ ID NO: 108, an HCDR2 amino acid sequence of SEQ ID NO: 109, and an HCDR3 amino acid sequence of SEQ ID NO: 110, and a VL comprising an LCDR1 amino acid sequence of SEQ ID NO: 111, an LCDR2 amino acid sequence of SEQ ID NO: 112, and an LCDR3 amino acid sequence of SEQ ID NO: 113;
(iv) a VH comprising an HCDR1 amino acid sequence of SEQ ID NO: 116, an HCDR2 amino acid sequence of SEQ ID NO: 117, and an HCDR3 amino acid sequence of SEQ ID NO: 118, and a VL comprising an LCDR1 amino acid sequence of SEQ ID NO: 119, an LCDR2 amino acid sequence of SEQ ID NO: 120, and an LCDR3 amino acid sequence of SEQ ID NO: 121;
(v) a VH comprising an HCDR1 amino acid sequence of SEQ ID NO: 124, an HCDR2 amino acid sequence of SEQ ID NO: 125, and an HCDR3 amino acid sequence of SEQ ID NO: 126, and a VL comprising an LCDR1 amino acid sequence of SEQ ID NO: 127, an LCDR2 amino acid sequence of SEQ ID NO: 128, and an LCDR3 amino acid sequence of SEQ ID NO: 129; or
(vi) a VH comprising an HCDR1 amino acid sequence of SEQ ID NO: 132, an HCDR2 amino acid sequence of SEQ ID NO: 133, and an HCDR3 amino acid sequence of SEQ ID NO: 134, and a VL comprising an LCDR1 amino acid sequence of SEQ ID NO: 135, an LCDR2 amino acid sequence of SEQ ID NO: 136, and an LCDR3 amino acid sequence of SEQ ID NO: 137.

6. The multispecific antibody molecule of claim 1, wherein:
(i) the VH comprises an amino acid sequence having at least 90% sequence identity to SEQ ID NO: 1, and the VL comprises an amino acid sequence having at least 90% sequence identity to SEQ ID NO: 2;
(ii) the VH comprises an amino acid sequence having at least 90% sequence identity to SEQ ID NO: 106, and the VL comprises an amino acid sequence having at least 90% sequence identity to SEQ ID NO: 107;
(iii) the VH comprises an amino acid sequence having at least 90% sequence identity to SEQ ID NO: 114, and the VL comprises an amino acid sequence having at least 90% sequence identity to SEQ ID NO: 115;
(iv) the VH comprises an amino acid sequence having at least 90% sequence identity to SEQ ID NO: 122, and the VL comprises an amino acid sequence having at least 90% sequence identity to SEQ ID NO: 123;
(v) the VH comprises an amino acid sequence having at least 90% sequence identity to SEQ ID NO: 130, and the VL comprises an amino acid sequence having at least 90% sequence identity to SEQ ID NO: 131; or
(vi) the VH comprises an amino acid sequence having at least 90% sequence identity to SEQ ID NO: 138, and the VL comprises an amino acid sequence having at least 90% sequence identity to SEQ ID NO: 139.

7. The multispecific antibody molecule of claim 1, wherein the at least one anti-TSHR antibody or antigen-binding domain comprises:
(i) a VH comprising an amino acid sequence according to SEQ ID NO: 1, and a VL comprising an amino acid sequence according to SEQ ID NO: 2;
(ii) a VH comprising an amino acid sequence according to SEQ ID NO: 106, and a VL comprising an amino acid sequence according to SEQ ID NO: 107;
(iii) a VH comprising an amino acid sequence according to SEQ ID NO: 114, and a VL comprising an amino acid sequence according to SEQ ID NO: 115;
(iv) a VH comprising an amino acid sequence according to SEQ ID NO: 122, and a VL comprising an amino acid sequence according to SEQ ID NO: 123;
(v) a VH comprising an amino acid sequence according to SEQ ID NO: 130, and a VL comprising an amino acid sequence according to SEQ ID NO: 131; or
(vi) a VH comprising an amino acid sequence according to SEQ ID NO: 138, and a VL comprising an amino acid sequence according to SEQ ID NO: 139.

8.-18. (canceled)

19. The multispecific antibody molecule of claim 1, wherein the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16.

20. The multispecific antibody molecule of claim 19, wherein the VH comprises an amino acid sequence having at least 90% sequence identity to SEQ ID NO: 9, and the VL comprises an amino acid sequence having at least 90% sequence identity to SEQ ID NO: 10.

21. The multispecific antibody molecule of claim 1, wherein the at least one anti-IGF-1R antibody or antigen-binding domain comprises a VH comprising an amino acid sequence according to SEQ ID NO: 9, and the VL comprising an amino acid sequence according to SEQ ID NO: 10.

22. The multispecific antibody molecule of claim 1, wherein:
(i)(a) the at least one anti-TSHR antibody or antigen-binding domain comprises a VH comprising an HCDR1 amino acid sequence of SEQ ID NO: 3, an HCDR2 amino acid sequence of SEQ ID NO: 4, and an HCDR3 amino acid sequence of SEQ ID NO: 5, and a VL comprising an LCDR1 amino acid sequence of SEQ ID NO: 6, an LCDR2 amino acid sequence of SEQ ID NO: 7, and an LCDR3 amino acid sequence of SEQ ID NO: 8; and
(b) the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16;
(ii)(a) the at least one anti-TSHR antibody or antigen-binding domain comprises a VH comprising an HCDR1 amino acid sequence of SEQ ID NO: 100, an HCDR2 amino acid sequence of SEQ ID NO: 101, and an HCDR3 amino acid sequence of SEQ ID NO: 102, and a VL comprising an LCDR1 amino acid sequence of SEQ ID NO: 103, an LCDR2 amino acid sequence of SEQ ID NO: 104, and an LCDR3 amino acid sequence of SEQ ID NO: 105; and
(b) the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16;
(iii)(a) the at least one anti-TSHR antibody or antigen-binding domain comprises a VH comprising an HCDR1 amino acid sequence of SEQ ID NO: 108, an HCDR2 amino acid sequence of SEQ ID NO: 109, and an HCDR3 amino acid sequence of SEQ ID NO: 110, and a VL comprising an LCDR1 amino acid sequence of SEQ ID NO: 111, an LCDR2 amino acid sequence of SEQ ID NO: 112, and an LCDR3 amino acid sequence of SEQ ID NO: 113; and
(b) the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16;
(iv)(a) the at least one anti-TSHR antibody or antigen-binding domain comprises a VH comprising an HCDR1 amino acid sequence of SEQ ID NO: 116, an HCDR2 amino acid sequence of SEQ ID NO: 117, and an HCDR3 amino acid sequence of SEQ ID NO: 118, and a VL comprising an LCDR1 amino acid sequence of SEQ ID NO: 119, an LCDR2 amino acid sequence of SEQ ID NO: 120, and an LCDR3 amino acid sequence of SEQ ID NO: 121; and
(b) the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16;
(v)(a) the at least one anti-TSHR antibody or antigen-binding domain comprises a VH comprising an HCDR1 amino acid sequence of SEQ ID NO: 124, an HCDR2 amino acid sequence of SEQ ID NO: 125, and an HCDR3 amino acid sequence of SEQ ID NO: 126, and a VL comprising an LCDR1 amino acid sequence of SEQ ID NO: 127, an LCDR2 amino acid sequence of SEQ ID NO: 128, and an LCDR3 amino acid sequence of SEQ ID NO: 129; and
(b) the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16; or
(vi)(a) the at least one anti-TSHR antibody or antigen-binding domain comprises a VH comprising an HCDR1 amino acid sequence of SEQ ID NO: 132, an HCDR2 amino acid sequence of SEQ ID NO: 133, and an HCDR3 amino acid sequence of SEQ ID NO: 134, and a VL comprising an LCDR1 amino acid sequence of SEQ ID NO: 135, an LCDR2 amino acid sequence of SEQ ID NO: 136, and an LCDR3 amino acid sequence of SEQ ID NO: 137; and
(b) the at least one anti-IGF-1R antibody or antigen-binding domain comprises a VH comprising an HCDR1 amino acid sequence of SEQ ID NO: 11, an HCDR2 amino acid sequence of SEQ ID NO: 12, and an HCDR3 amino acid sequence of SEQ ID NO: 13, and a VL comprising an LCDR1 amino acid sequence of SEQ ID NO: 14, an LCDR2 amino acid sequence of SEQ ID NO: 15, and an LCDR3 amino acid sequence of SEQ ID NO: 16.

23. The multispecific antibody molecule of claim 22, wherein:
(i)(a) the at least one anti-TSHR antibody or antigen-binding domain comprises a VH comprising an amino acid sequence having at least 90% sequence identity to SEQ ID NO: 1, and a VL comprising an amino acid sequence having at least 90% sequence identity to SEQ ID NO: 2; and
(b) the at least one anti-IGF-1R antibody or antigen-binding domain comprises a VH comprising an amino acid sequence having at least 90% sequence identity to SEQ ID NO: 9, and a VL comprising an amino acid sequence having at least 90% sequence identity to SEQ ID NO: 10;
(ii)(a) the at least one anti-TSHR antibody or antigen-binding domain comprises a VH comprising an amino acid sequence having at least 90% sequence identity to SEQ ID NO: 106, and a VL comprising an amino acid sequence having at least 90% sequence identity to SEQ ID NO: 107; and
(b) the at least one anti-IGF-1R antibody or antigen-binding domain comprises a VH comprising an amino acid sequence having at least 90% sequence identity to SEQ ID NO: 9, and a VL comprising an amino acid sequence having at least 90% sequence identity to SEQ ID NO: 10;
(iii)(a) the at least one anti-TSHR antibody or antigen-binding domain comprises a VH comprising an amino acid sequence having at least 90% sequence identity to SEQ ID NO: 114, and a VL comprising an amino acid sequence having at least 90% sequence identity to SEQ ID NO: 115; and
(b) the at least one anti-IGF-1R antibody or antigen-binding domain comprises a VH comprising an amino acid sequence having at least 90% sequence identity to SEQ ID NO: 9, and a VL comprising an amino acid sequence having at least 90% sequence identity to SEQ ID NO: 10;
(iv)(a) the at least one anti-TSHR antibody or antigen-binding domain comprises a VH comprising an amino acid sequence having at least 90% sequence identity to SEQ ID NO: 122, and a VL comprising an amino acid sequence having at least 90% sequence identity to SEQ ID NO: 123; and
(b) the at least one anti-IGF-1R antibody or antigen-binding domain comprises a VH comprising an amino acid sequence having at least 90% sequence identity to SEQ ID NO: 9, and a VL comprising an amino acid sequence having at least 90% sequence identity to SEQ ID NO: 10;
(v)(a) the at least one anti-TSHR antibody or antigen-binding domain comprises a VH comprising an amino acid sequence having at least 90% sequence identity to SEQ ID NO: 130, and a VL comprising an amino acid sequence having at least 90% sequence identity to SEQ ID NO: 131; and
(b) the at least one anti-IGF-1R antibody or antigen-binding domain comprises a VH comprising an amino acid sequence having at least 90% sequence identity to SEQ ID NO: 9, and a VL comprising an amino acid sequence having at least 90% sequence identity to SEQ ID NO: 10; or
(vi)(a) the at least one anti-TSHR antibody or antigen-binding domain comprises a VH comprising an amino acid sequence having at least 90% sequence identity to SEQ ID NO: 138, and a VL comprising an amino acid sequence having at least 90% sequence identity to SEQ ID NO: 139; and
(b) the at least one anti-IGF-1R antibody or antigen-binding domain comprises a VH comprising an amino acid sequence having at least 90% sequence identity to SEQ ID NO: 9, and a VL comprising an amino acid sequence having at least 90% sequence identity to SEQ ID NO: 10.

24. The multispecific antibody molecule of claim 1, wherein:
(i)(a) the at least one anti-TSHR antibody or antigen-binding domain comprises a VH comprising an amino acid sequence according to SEQ ID NO: 1, and a VL comprising an amino acid sequence according to SEQ ID NO: 2; and
(b) the at least one anti-IGF-1R antibody or antigen-binding domain comprises a VH comprising an amino acid sequence according to SEQ ID NO: 9, and a VL comprising an amino acid sequence according to SEQ ID NO: 10;
(ii)(a) the at least one anti-TSHR antibody or antigen-binding domain comprises a VH comprising an amino acid sequence according to SEQ ID NO: 106, and a VL comprising an amino acid sequence according to SEQ ID NO: 107; and
(b) the at least one anti-IGF-1R antibody or antigen-binding domain comprises a VH comprising an amino acid sequence according to SEQ ID NO: 9, and a VL comprising an amino acid sequence according to SEQ ID NO: 10;
(iii)(a) the at least one anti-TSHR antibody or antigen-binding domain comprises a VH comprising an amino acid sequence according to SEQ ID NO: 114, and a VL comprising an amino acid sequence according to SEQ ID NO: 115; and
(b) the at least one anti-IGF-1R antibody or antigen-binding domain comprises a VH comprising an amino acid sequence according to SEQ ID NO: 9, and a VL comprising an amino acid sequence according to SEQ ID NO: 10;
(iv)(a) the at least one anti-TSHR antibody or antigen-binding domain comprises a VH comprising an amino acid sequence according to SEQ ID NO: 122, and a VL comprising an amino acid sequence according to SEQ ID NO: 123; and
(b) the at least one anti-IGF-1R antibody or antigen-binding domain comprises a VH comprising an amino acid sequence according to SEQ ID NO: 9, and a VL comprising an amino acid sequence according to SEQ ID NO: 10;
(v)(a) the at least one anti-TSHR antibody or antigen-binding domain comprises a VH comprising an amino acid sequence according to SEQ ID NO: 130, and a VL comprising an amino acid sequence according to SEQ ID NO: 131; and
(b) the at least one anti-IGF-1R antibody or antigen-binding domain comprises a VH comprising an amino acid sequence according to SEQ ID NO: 9, and a VL comprising an amino acid sequence according to SEQ ID NO: 10; or
(vi)(a) the at least one anti-TSHR antibody or antigen-binding domain comprises a VH comprising an amino acid sequence according to SEQ ID NO: 138, and a VL comprising an amino acid sequence according to SEQ ID NO: 139; and
(b) the at least one anti-IGF-1R antibody or antigen-binding domain comprises a VH comprising an amino acid sequence according to SEQ ID NO: 9, and a VL comprising an amino acid sequence according to SEQ ID NO: 10.

25.-32. (canceled)

33. The multispecific antibody molecule of claim 1, wherein the at least one anti-TSHR antibody or antigen-binding domain is a recombinant scFv (single chain fragment variable) antibody, Fab fragment, F(ab′)2 fragment, or Fv fragment, and/or the at least one anti-IGF-1R antibody or antigen-binding domain is a recombinant scFv antibody, Fab fragment, F(ab′)2 fragment, or Fv fragment.

34. The multispecific antibody molecule of claim 1, wherein the multispecific antibody molecule is an IgG, or a recombinant IgG antibody or antibody fragment comprising an Fc portion mutated to alter FcR interactions, to increase half-life, and/or to increase therapeutic efficacy, and/or glycan modified to alter FcR interactions.

35. The multispecific antibody molecule of claim 34, wherein the Fc portion mutated to alter FcR interactions, to increase half-life, and/or to increase therapeutic efficacy comprises a LALA, LALA PG, N297, GASD/ALIE, DHS, YTE or LS mutation.

36. The multispecific antibody molecule of claim 34, wherein the glycan modified to alter FcR interactions comprises enzymatic or chemical addition or removal of glycans, or expression in a cell line engineered with a defined glycosylating pattern.

37. A pharmaceutical composition comprising the multispecific antibody molecule of claim 1, and one or more pharmaceutically acceptable excipients, diluents and/or carriers.

38. (canceled)

39. A method of treating a condition or disorder in which aberrant stimulation of TSHR and/or aberrant signalling of IGF-1R occurs in a subject in need thereof, comprising administering to the subject the multispecific antibody molecule of claim 1, thereby treating the condition or disorder in the subject.

40. The method of claim 39, wherein the condition or disorder is thyroid-associated ophthalmopathy (TAO).
